SEQUENCE LISTING (104 sequences)

SEQ ID NO: 1 - protein, 9 aa, Artificial Sequence
  Synthetic peptide
CKGGRAKDC

SEQ ID NO: 2 - protein, 9 aa, Artificial Sequence
  Synthetic peptide
CWELGGGPC

SEQ ID NO: 3 - protein, 9 aa, Artificial Sequence
  Synthetic peptide
CHVLGGGPC

SEQ ID NO: 4 - protein, 9 aa, Artificial Sequence
  Synthetic peptide
CVQGGGGPC

SEQ ID NO: 5 - protein, 9 aa, Artificial Sequence
  Synthetic peptide
CWKLGGGPC

SEQ ID NO: 6 - protein, 9 aa, Artificial Sequence
  Synthetic peptide
CPGGGLVHC

SEQ ID NO: 7 - protein, 5 aa, Artificial Sequence
  Synthetic peptide
CARAC

SEQ ID NO: 8 - protein, 16 aa, Artificial Sequence
  Synthetic peptide
LFPDFFTRVAYVDWIR

SEQ ID NO: 9 - protein, 36 aa, Artificial Sequence
  Synthetic peptide
LVNVVLGAHNVRTQEPTQQHFSVAQVFLNNYDAENK

SEQ ID NO: 10 - protein, 21 aa, Artificial Sequence
  Synthetic peptide
IVGGHEAQPHSRPYMASLQMR

SEQ ID NO: 11 - protein, 256 aa, Artificial Sequence
  Synthetic peptide
MAHRPPSPALASVLLALLISGAARAAEIVGGHEAQPHSRPYMASLQMRGNPGSHFCGGTLIHPSFVLTAPHCLRDIPQRLVNVVLGAHNVRTQEPTQQHFSVAQVFLNNYDAENKLNDILLIQLSSPANLSASVTSVQLPQQDQPVPPHGTQCLAMGWGRVGAHDPPAQVLQELNVTVVTFFCRPHNICTFVPRRKAGICCFGDSGGPLICDGIIQGIDSFVIWGCATRLFPDFFTRVAYDWIRSTLRRVEAKGRP

SEQ ID NO: 12 - protein, 9 aa, Artificial Sequence
  Synthetic peptide
CXXXXXXXC

SEQ ID NO: 13 - protein, 7 aa, Artificial Sequence
  Synthetic peptide
WKLGGGP

SEQ ID NO: 14 - protein, 7 aa, Artificial Sequence
  Synthetic peptide
ASGRRES

SEQ ID NO: 15 - protein, 7 aa, Artificial Sequence
  Synthetic peptide
HQKLLVA

SEQ ID NO: 16 - protein, 7 aa, Artificial Sequence
  Synthetic peptide
GRRSRDE

SEQ ID NO: 17 - protein, 7 aa, Artificial Sequence
  Synthetic peptide
GLVALAR

SEQ ID NO: 18 - protein, 7 aa, Artificial Sequence
  Synthetic peptide
AGSGSVL

SEQ ID NO: 19 - protein, 6 aa, Artificial Sequence
  Synthetic peptide
LLGKGR

SEQ ID NO: 20 - protein, 6 aa, Artificial Sequence
  Synthetic peptide
LASGRV

SEQ ID NO: 21 - protein, 7 aa, Artificial Sequence
  Synthetic peptide
SPAWGVR

SEQ ID NO: 22 - protein, 7 aa, Artificial Sequence
  Synthetic peptide
NLSGRRA

SEQ ID NO: 23 - protein, 6 aa, Artificial Sequence
  Synthetic peptide
GRRGEV

SEQ ID NO: 24 - protein, 7 aa, Artificial Sequence
  Synthetic peptide
GGRAHGI

SEQ ID NO: 25 - protein, 7 aa, Artificial Sequence
  Synthetic peptide
GLALGGF

SEQ ID NO: 26 - protein, 7 aa, Artificial Sequence
  Synthetic peptide
YSAMAGG

SEQ ID NO: 27 - protein, 7 aa, Artificial Sequence
  Synthetic peptide
EAVSGGP

SEQ ID NO: 28 - protein, 7 aa, Artificial Sequence
  Synthetic peptide
DLVASGV

SEQ ID NO: 29 - protein, 7 aa, Artificial Sequence
  Synthetic peptide
PAVTGGQ

SEQ ID NO: 30 - protein, 7 aa, Artificial Sequence
  Synthetic peptide
GLAGGVT

SEQ ID NO: 31 - protein, 7 aa, Artificial Sequence
  Synthetic peptide
HYAAGVV

SEQ ID NO: 32 - protein, 7 aa, Artificial Sequence
  Synthetic peptide
GVASGVW

SEQ ID NO: 33 - protein, 7 aa, Artificial Sequence
  Synthetic peptide
RRVNSAV

SEQ ID NO: 34 - protein, 7 aa, Artificial Sequence
  Synthetic peptide
VVNNAQA

SEQ ID NO: 35 - protein, 7 aa, Artificial Sequence
  Synthetic peptide
APALYDV

SEQ ID NO: 36 - protein, 7 aa, Artificial Sequence
  Synthetic peptide
VIAGGRA

SEQ ID NO: 37 - protein, 7 aa, Artificial Sequence
  Synthetic peptide
GAFRGVK

SEQ ID NO: 38 - protein, 7 aa, Artificial Sequence
  Synthetic peptide
RFHGVTS

SEQ ID NO: 39 - protein, 7 aa, Artificial Sequence
  Synthetic peptide
RMLVGEG

SEQ ID NO: 40 - protein, 7 aa, Artificial Sequence
  Synthetic peptide
GIVVGEN

SEQ ID NO: 41 - protein, 7 aa, Artificial Sequence
  Synthetic peptide
VLVGEGG

SEQ ID NO: 42 - protein, 7 aa, Artificial Sequence
  Synthetic peptide
IVVSESV

SEQ ID NO: 43 - protein, 7 aa, Artificial Sequence
  Synthetic peptide
PVAEGTS

SEQ ID NO: 44 - protein, 7 aa, Artificial Sequence
  Synthetic peptide
VMLASAL

SEQ ID NO: 45 - protein, 7 aa, Artificial Sequence
  Synthetic peptide
RELAGQG

SEQ ID NO: 46 - protein, 7 aa, Artificial Sequence
  Synthetic peptide
KRLGSLG

SEQ ID NO: 47 - protein, 6 aa, Artificial Sequence
  Synthetic peptide
EVGSAV

SEQ ID NO: 48 - protein, 7 aa, Artificial Sequence
  Synthetic peptide
ELAAGIG

SEQ ID NO: 49 - protein, 7 aa, Artificial Sequence
  Synthetic peptide
DLAPAFE

SEQ ID NO: 50 - protein, 7 aa, Artificial Sequence
  Synthetic peptide
SGLSASH

SEQ ID NO: 51 - protein, 7 aa, Artificial Sequence
  Synthetic peptide
RALSGAY

SEQ ID NO: 52 - protein, 7 aa, Artificial Sequence
  Synthetic peptide
ELLGHLE

SEQ ID NO: 53 - protein, 7 aa, Artificial Sequence
  Synthetic peptide
TLGGRLG

SEQ ID NO: 54 - protein, 7 aa, Artificial Sequence
  Synthetic peptide
LSGHVQI

SEQ ID NO: 55 - protein, 7 aa, Artificial Sequence
  Synthetic peptide
VSLGLGA

SEQ ID NO: 56 - protein, 7 aa, Artificial Sequence
  Synthetic peptide
VVMGLRG

SEQ ID NO: 57 - protein, 7 aa, Artificial Sequence
  Synthetic peptide
VLLGKWG

SEQ ID NO: 58 - protein, 7 aa, Artificial Sequence
  Synthetic peptide
HLGLTRL

SEQ ID NO: 59 - protein, 7 aa, Artificial Sequence
  Synthetic peptide
VGLVKGV

SEQ ID NO: 60 - protein, 7 aa, Artificial Sequence
  Synthetic peptide
SALMSVG

SEQ ID NO: 61 - protein, 7 aa, Artificial Sequence
  Synthetic peptide
QLLDAAL

SEQ ID NO: 62 - protein, 7 aa, Artificial Sequence
  Synthetic peptide
HFETGGY

SEQ ID NO: 63 - protein, 7 aa, Artificial Sequence
  Synthetic peptide
KGLKGAR

SEQ ID NO: 64 - protein, 7 aa, Artificial Sequence
  Synthetic peptide
LYTVGLG

SEQ ID NO: 65 - protein, 7 aa, Artificial Sequence
  Synthetic peptide
MQLKSGI

SEQ ID NO: 66 - protein, 7 aa, Artificial Sequence
  Synthetic peptide
SRTAVLG

SEQ ID NO: 67 - protein, 7 aa, Artificial Sequence
  Synthetic peptide
KGLKGAR

SEQ ID NO: 68 - protein, 7 aa, Artificial Sequence
  Synthetic peptide
TGSRLSG

SEQ ID NO: 69 - protein, 7 aa, Artificial Sequence
  Synthetic peptide
SGTALDG

SEQ ID NO: 70 - protein, 7 aa, Artificial Sequence
  Synthetic peptide
DLMAISG

SEQ ID NO: 71 - protein, 5 aa, Artificial Sequence
  Synthetic peptide
ALPGR

SEQ ID NO: 72 - protein, 7 aa, Artificial Sequence
  Synthetic peptide
SSVISGK

SEQ ID NO: 73 - protein, 7 aa, Artificial Sequence
  Synthetic peptide
SSLLNGS

SEQ ID NO: 74 - protein, 7 aa, Artificial Sequence
  Synthetic peptide
WVMLSGN

SEQ ID NO: 75 - protein, 7 aa, Artificial Sequence
  Synthetic peptide
LDGYLKR

SEQ ID NO: 76 - protein, 7 aa, Artificial Sequence
  Synthetic peptide
TSVSLAV

SEQ ID NO: 77 - protein, 7 aa, Artificial Sequence
  Synthetic peptide
LPGLARG

SEQ ID NO: 78 - protein, 6 aa, Artificial Sequence
  Synthetic peptide
ALAESG

SEQ ID NO: 79 - protein, 7 aa, Artificial Sequence
  Synthetic peptide
SPAWGVR

SEQ ID NO: 80 - protein, 6 aa, Artificial Sequence
  Synthetic peptide
LAVGRV

SEQ ID NO: 81 - protein, 7 aa, Artificial Sequence
  Synthetic peptide
LLMRGRS

SEQ ID NO: 82 - protein, 7 aa, Artificial Sequence
  Synthetic peptide
GLQRGRT

SEQ ID NO: 83 - protein, 7 aa, Artificial Sequence
  Synthetic peptide
GLASGTA

SEQ ID NO: 84 - protein, 7 aa, Artificial Sequence
  Synthetic peptide
WLPKGRG

SEQ ID NO: 85 - protein, 7 aa, Artificial Sequence
  Synthetic peptide
VIAGGRA

SEQ ID NO: 86 - protein, 7 aa, Artificial Sequence
  Synthetic peptide
NLSGRRA

SEQ ID NO: 87 - protein, 7 aa, Artificial Sequence
  Synthetic peptide
QVKGRQG

SEQ ID NO: 88 - protein, 7 aa, Artificial Sequence
  Synthetic peptide
ASGRRES

SEQ ID NO: 89 - protein, 6 aa, Artificial Sequence
  Synthetic peptide
SGRRGE

SEQ ID NO: 90 - protein, 7 aa, Artificial Sequence
  Synthetic peptide
GRRSRDE

SEQ ID NO: 91 - protein, 7 aa, Artificial Sequence
  Synthetic peptide
PRRAGES

SEQ ID NO: 92 - protein, 7 aa, Artificial Sequence
  Synthetic peptide
QGRAQDL

SEQ ID NO: 93 - protein, 7 aa, Artificial Sequence
  Synthetic peptide
GGRAHGI

SEQ ID NO: 94 - protein, 7 aa, Artificial Sequence
  Synthetic peptide
IRASDSL

SEQ ID NO: 95 - protein, 7 aa, Artificial Sequence
  Synthetic peptide
RRAEGDA

SEQ ID NO: 96 - protein, 7 aa, Artificial Sequence
  Synthetic peptide
LVQYELL

SEQ ID NO: 97 - protein, 7 aa, Artificial Sequence
  Synthetic peptide
AGSGSVL

SEQ ID NO: 98 - protein, 7 aa, Artificial Sequence
  Synthetic peptide
AEGGTIN

SEQ ID NO: 99 - protein, 7 aa, Artificial Sequence
  Synthetic peptide
REGSVGS

SEQ ID NO: 100 - protein, 7 aa, Artificial Sequence
  Synthetic peptide
HDGAVRS

SEQ ID NO: 101 - protein, 7 aa, Artificial Sequence
  Synthetic peptide
VSVLDTA

SEQ ID NO: 102 - protein, 7 aa, Artificial Sequence
  Synthetic peptide
ATLIGQN

SEQ ID NO: 103 - protein, 7 aa, Artificial Sequence
  Synthetic peptide
LARGLFG

SEQ ID NO: 104 - protein, 7 aa, Artificial Sequence
  Synthetic peptide
TAGAPRP

(19) United States
(12) Patent Application Publication
(10) Pub. No.: US 2014/0356285 A1
(43) Pub. Date: Dec. 4, 2014

(54) COMPOSITIONS AND METHODS RELATED TO TISSUE TARGETING

(71) Applicant: THE BOARD OF REGENTS OF THE UNIVERSITY OF TEXAS SYSTEM, Austin, TX (US)

(72) Inventors: Wadih Arap, Albuquerque, NM (US); Renata Pasqualini, Albuquerque, NM (US); Mikhail G. Kolonin, Houston, TX (US)

(73) Assignee: THE BOARD OF REGENTS OF THE UNIVERSITY OF TEXAS SYSTEM, Austin, TX (US)

(21) Appl. No.: 14/346,421

(22) PCT Filed: Sep. 20, 2012
(86) PCT No.: PCT/US12/56333

§ 371 Date: Mar. 21, 2014

Related U.S. Application Data
(60) Provisional application No. 61/538,322, filed on Sep. 23, 2011.

(51) Int. Cl. A61K 47/48

(52) U.S. Cl. CPC A61K 47/48246
USPC 424/1.69; 530/329; 514/21.7; 514/21.8; 424/130.1; 424/193.1

(57) ABSTRACT

Systems and reagents for identification, characterization and/or targeting of particular tissue or cell markers are disclosed. Methods and compositions for in vivo and in vitro targeting of particular targets are also disclosed. Peptides are employed for targeted delivery of therapeutic or imaging agents.

The application claims priority to U.S. Provisional Patent Application No. 61/538,322 filed Sep. 23, 2011, which is incorporated herein by reference in its entirety.

### STATEMENT REGARDING FEDERALLY SPONSORED RESEARCH OR DEVELOPMENT

The invention was made with government support under Grant No. P50CA90270 (PP-2), P50CA90270 (PP-4), and R33CA103030 awarded by the National Institutes of Health/National Cancer Institute. The government has certain rights in the invention.

### TECHNICAL FIELD

The present invention generally concerns the fields of cell biology, molecular biology, and medicine. In particular aspects, the field of the invention includes peptides that target certain tissues, for example for diagnostic and/or therapeutic purposes.

### BACKGROUND OF THE INVENTION

Targeting peptides that exhibit selective and/or specific binding for particular cells or tissues, such as adipose tissues or cancer cells, have a variety of uses. Targeting peptides against adipose tissue could be used to control obesity and related conditions, for example. Adipose-targeting peptides would also be of potential use to treat HIV related adipose malformations such as lipodystrophia and/or hyperlipidemia (see, e. g., Zhang et al., J. Clin. Endocrin. Metab. 84: 4274-77, 1999; Jain et al., Antiviral Res. 51: 151-177, 2001; Raolin et al., Prog. Lipid Res. 41: 27-65, 2002). Presently available methods for control of weight include dieting and surgical procedures, but these often exhibit adverse effects and may not result in long-term weight loss. Dieting includes both popular (fad) diets and the use of weight loss and appetite supplements. Fad diets are only good for short-term weight loss and do not achieve long-term weight control. They are often unhealthy, since many important nutrients are missing from the diet. In addition, rapid weight loss can result in dehydration. After losing weight, the dieters typically return to their original eating habits. This often results in weight gain that can exceed the subject's weight before dieting (yo yo effect).

Appetite suppressants may also have adverse effects. These supplements can also lead to more serious problems like eating disorders. Weight control through use of such supplements is ineffective, with only limited weight loss achieved. Effective drugs for controlling weight, such as fenfluramine, were withdrawn from the market due to cardiotoxicity. Surgical methods for weight reduction, such as liposuction and gastric bypass surgery, have many risks. Liposuction removes subcutaneous fat through a suction tube inserted into a small incision in the skin. Risks and complications may include scarring, bleeding, infection, change in skin sensation, pulmonary complications, skin loss, chronic pain, etc. In gastric bypass surgery, the patient has to go through the rest of his or her life with a drastically altered stomach that can hold just two or three ounces of food. Side effects may include nausea, diarrhea, bleeding, infection, bowel blockage caused by scar tissue, hernia and adverse reactions to general anesthesia. The most serious potential risk is leakage of fluid from the stomach or intestines, which may result in abdominal infection and the need for a second surgery. None of the presently available methods for weight control is satisfactory and a need exists for improved methods of weight loss and control.

Another adipose related disease state is lipodystrophy syndrome (s) related to HIV infection (e.g., Jain et al., Antiviral Res. 51: 151-177, 2001). Mortality rates from HIV infection have decreased substantially following use of highly active antiretroviral therapy (HAART) (Id.) However, treatment with protease inhibitors as part of the HAART protocol appears to result in a number of lipid-related symptoms, such as hyperlipidemia, fat redistribution with accumulation of abdominal and cervical fat, diabetes mellitus and insulin resistance (Jain et al., 2001; Yanovski et al., J. Clin. Endocrin. Metab. 84: 1925-1931; Raulin et al., Prog. Lipid Res. 41: 27-65, 2002). A need exists in the art for more effective methods of treating HTV related lypodystrophy.

Attachment of therapeutic agents to targeting peptides has resulted in the selective delivery of the agent to a desired organ, tissue or cell type in a mouse model system. Targeted delivery of chemotherapeutic agents and proapoptotic peptides to receptors located in tumor angiogenic vasculature resulted in a marked increase in therapeutic efficacy and a decrease in systemic toxicity in tumor-bearing mouse models (Arap et al., 1998a, 1998b; Ellerby et al., 1999). However, the targeted delivery of anti-cancer agents in humans has not yet been demonstrated. The targeted receptors reported in previous studies may be present in angiogenic normal tissues as well as in tumor tissues and may or may not be of use in distinguishing between normal tissues, non-metastatic cancers and metastatic cancer. A need exists for tumor targeting peptides that are selective against human cancers, as well as for targeting peptides that can distinguish between metastatic and non-metastatic human cancers.

Attempts have been made to target delivery of gene therapy vectors to specific organs, tissues or cell types in vivo. Directing such vectors to the site of interest would enhance therapeutic effects and diminish adverse systemic immunologic responses. A need also exists to identify receptor-ligand pairs in organs, tissues or cell types. Previous attempts to identify targeted receptors and ligands binding to receptors have largely targeted a single ligand at a time for investigation. Identification of previously unknown receptors and previously uncharacterized ligands has been a very slow and laborious process. Such novel receptors and ligands may provide the basis for new therapies for a variety of disease states, such as cancer and/or metastatic cancer.

In a variety of contexts, it is desirable to target cells and tissues of a particular nature, such as adipose cells or cancer cells, for example.

### BRIEF SUMMARY OF THE INVENTION

The present invention encompasses the recognition that molecules differentially expressed in certain cells and tissues are attractive targets for therapy. Among other things, the present invention provides systems and reagents for identification, characterization and/or targeting of cell markers.

In some embodiments, the present invention provides a targeting agent characterized in that it interacts specifically with a targeted entity generally present on cells having certain receptors, and in specific embodiments it is internalized into the cells. In some embodiments, the targeted entity may be a receptor or fragment thereof. In some embodiments, the present invention provides a method of delivering a targeting agent to a targeted entity site, including steps of delivering to the targeted entity site a targeting agent characterized in that it interacts specifically with the targeted entity, and wherein the targeting agent is internalized upon interacting with said targeted entity.

In some embodiments, the present invention provides a method of identifying targeting agents, including steps of providing a system including particular cells or tissues; providing a plurality of candidate targeting agents; contacting the system with members from the plurality; and determining that members of the plurality bind to and are internalized by the cells, and not by other types of cells in the system. In some embodiments, the system comprises an organism (e.g., a human).

The present invention solves a long-standing need in the art by providing compositions and methods for identifying and/or using targeting peptides that are selective for specific organs, tissues, and/or cell types. Provided systems and reagents may be employed in vitro or in vivo in any mammal, including human, mouse, rat, dog, cat, horse, sheep, goat, pig, or cow, although certain cases the system is avian.

In some embodiments, the methods may comprise contacting a targeting peptide to an organ or tissue and/or cell containing a receptor of interest.

In some embodiments, one or more ligands for a receptor of interest may be identified by the methods and compositions described herein. For example, in some embodiments, one or more targeting peptides that mimic part or all of a naturally occurring ligand may be identified by phage display and biopanning in vivo or in vitro. A naturally occurring ligand may be identified by homology with a single targeting peptide that binds to the receptor, or a consensus motif of sequences that bind to the receptor.

In certain embodiments, the targeting peptides of the present invention are of use for the selective delivery of agents (e.g., active agents), including but not limited to drugs, antibodies, polynucleotides, gene therapy vectors and/or fusion proteins, to specific organs, tissues or cell types, including adipose tissue or cancer tissues or cells, for example. The skilled artisan will appreciate that the scope of the claimed methods of use include any non-disease or disease state. For example, in some embodiments, particular cells are present in a healthy subject. In some embodiments, particular cells are present in an individual suffering from cancer or a disease, disorder or condition associated with white adipose tissue. In some embodiments, such diseases may be treated by targeted delivery of a therapeutic agent to a desired endothelial tissue and/or cell type. Such disease states include, but are not limited to, obesity or cancer, including non-metastatic cancer.

In some embodiments, there are methods and reagents concerning one or more peptides that specifically target receptors on adipose tissue or vascular tissue associated with adipose tissue. The methods and reagents may concern the treatment, diagnosis, and research of related to a variety of diseases, disorders, or conditions, but in certain embodiments they are useful for obesity, being overweight, metabolic conditions associated with obesity or being overweight, or cancer diagnosis, treatment, or research thereof. In some embodiments, there are methods and reagents related to the CKGGRAKDC (SEQ ID NO:1) peptide (with or without the cysteine residue on the N-terminus and/or C-terminus), any functional fragments thereof, or any derivatives or functional analogs of any such peptides or fragments.

In some embodiments, there are methods and reagents concerning one or more peptides that specifically target receptors on cancer cells. The methods and reagents concern the treatment, diagnosis, and research of cancer, in specific embodiments. In some embodiments, there are methods and reagents related to one or more of the CWELGGGPC (SEQ ID NO:2), CHVLGGGPC (SEQ ID NO:3), and/or CVQGGGGPC (SEQ ID NO:4) peptides (with or without the cysteine residue on the N-terminus and/or C-terminus), any functional fragments thereof, or any derivatives or functional analogs of any such peptides or fragments.

In certain embodiments, an isolated peptide is attached to a molecule. In some embodiments, the attachment is a covalent attachment. In some embodiments, the molecule is a drug, a chemotherapeutic agent, a radioisotope, a pro-apoptosis agent, an anti-angiogenic agent, a hormone, an anti-hormone, a cytokine, a growth factor, a cytotoxic agent, a peptide, a protein, an antibiotic, an antibody, a Fab fragment of an antibody, a survival factor, an anti-apoptotic factor, a hormone antagonist, an imaging agent, a nucleic acid (e.g., DNA, RNA, siRNA, miRNA, or antisense RNA), a lipid, or an antigen. It will be appreciated that molecules within the scope of the present invention include virtually any molecule that may be attached to a targeting peptide and administered to a subject.

In some embodiments, the pro-aptoptosis agent is gramicidin, magainin, mellitin, defensin, or cecropin. In some embodiments, the anti-angiogenic agent is angiostatin5, pigment epithelium-drived factor, angiotensin, laminin peptides, fibronectin peptides, plasminogen activator inhibitors, tissue metalloproteinase inhibitors, interferons, interleukin 12, platelet factor 4, IP-10, Gro-13, thrombospondin, 2-methoxyoestradiol, proliferin-related protein, carboxiamidotriazole, CM101, Marimastat, pentosan polysulphate, angiopoletin 2 (Regeneron), interferon-alpha, herbimycin A, PNU145156E, 16K prolactin fragment, linomide, thalidomide, pentoxifylline, genistein, TNP470, endostatin, paclitaxel, docetaxel, polyamines, a proteasome inhibitor, a kinase inhibitor, a signaling inhibitor (SU5416, SU6668, Sugen, South San Francisco, Calif.), accutin, cidofovir, vincristine, bleomycin, AGM-1470, platelet factor 4 or minocycline. In some embodiments, the cytokine is interleukin 1 (IL-1), IL-2, IL-5, IL-10, IL-11, IL-12, IL-18, interferon-γ (IF-γ), IF-α, IF-β, tumor necrosis factor-.alpha. (TNF-α), or GM-CSF (granulocyte macrophage colony stimulating factor). Such examples are representative only and are not intended to exclude other pro-apoptosis agents, anti-angiogenic agents or cytokines known in the art.

In some embodiments, the isolated peptide is attached to a macromolecular complex. In some embodiments, the attachment is a covalent attachment. In some embodiments, the macromolecular complex is a virus, a bacteriophage, a bacterium, a liposome, a microparticle, a nanoparticle, a magnetic bead, a yeast cell, a mammalian cell, a cell, a quantum dot, an expression vector, or a microdevice. These are representative examples only. Macromolecular complexes within the scope of the present invention include virtually any macromolecular complex that may be attached to a targeting peptide and administered to a subject. In some embodiments, the isolated peptide is attached to a eukaryotic expression vector.

In some embodiments, the present invention provides compositions and methods of use of targeting peptides against adipose cells and/or cancer cells. The cancer cell may or may not be in a solid tumor. Such targeting peptides may be attached to therapeutic agents, including but not limited to molecules or macromolecular assemblages and administered to a subject in need of treatment. Without wishing to be bound by any particular theories, it is thought that targeted delivery of therapeutic agents provides a significant improvement over the prior art for increasing the delivery of the agent to a target organ, cell, or tissue, while decreasing the inadvertent delivery of the agent to unaffected organs and tissues of the subject.

Some embodiments of the present invention concern compositions and methods of use of tumor targeting peptides against cancers. Tumor targeting peptides identified by the methods disclosed in the instant application may be attached to therapeutic agents, including but not limited to molecules or macromolecular assemblages and administered to a subject with cancer, providing for increased efficacy and decreased systemic toxicity of the therapeutic agent. Therapeutic agents within the scope of the present invention include but are not limited to chemotherapeutic agents, radioisotopes, pro-apoptosis agents, cytotoxic agents, cytostatic agents and gene therapy vectors. Targeted delivery of such therapeutic agents to tumors provides a significant improvement over the prior art for increasing the delivery of the agent to the tumor, while decreasing the inadvertent delivery of the agent to normal organs and tissues of the subject.

In some embodiments, provided targeting agents interact specifically with a targeted entity present on particular cells, such as adipose cells or cancer cells, and the targeting agent may be internalized into the cells. In some embodiments, the targeted entity is a vascular receptor or fragment thereof. In some embodiments the targeted entity is or comprises prohibitin or human leukocyte proteinase-3. It will be appreciated that the targeting agent may be of any structure. For example, in some embodiments the agent is or comprises a small molecule or a peptide. The targeting agent may comprise a peptide having amino acid sequence that has at least 80%, 85%, 90%, 95%, 98%, 99%, or 100% overall identity with a portion of ANXA2 or RAGE that is at least 3 amino acids in length, wherein the pan-endothelial targeting agent is not integrin alpha 4 or cathespin B. In some embodiments, the agent includes an amino acid moiety as set forth in SEQ ID NOs:1-6, for example. In some embodiments, the peptide is cyclic.

In some embodiments, the targeted entity comprises an antibody or a fragment thereof. In some embodiments, the targeting agent comprises a targeting agent linked with an active agent, such as a diagnostic agent and/or a therapeutic agent. For example, the active agent may comprise a nucleic acid agent, such as DNA, RNA, or a combination thereof. In some embodiments the agent comprises siRNA, miRNA, or antisense RNA.

In some embodiments, the active agent is or comprises an imaging agent, including a radioisotope, for example. Exemplary radioisotopes include, but are not limited to ⁶⁴Cu, ¹¹¹In, ²¹³Bi, ¹⁰³Pd, ¹³³Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ¹¹⁷Sn, ¹⁸⁶Re, ¹⁶⁶Ho and ¹⁸⁸Re. Other examples of imaging agents include one or more of an enzyme, a fluorescent label, a near infrared label, a luminescent label, a bioluminescent label, a magnetic label, and biotin.

In some embodiments of the invention, the active agent is or comprises an antibody agent. In some embodiments, the antibody agent is or comprises a monoclonal antibody, a polyclonal antibody, an Fc portion, an Fab, an ScFv, and a single domain antibody.

In some embodiments, the targeting agent includes a peptide that is at least part of an active agent. It will be appreciated that the peptide may be of any length. In some embodiments the peptide is 3-30 residues, 3-25 residues, 3-20 residues, 3-15 residues, 3-10 residues, 4-30 residues, 4-25 residues, 4-20 residues, 4-15 residues, 4-10 residues, 5-30 residues, 5-25 residues, 5-20 residues, 5-15 residues, 5-10 residues, 6-30 residues, 6-25 residues, 6-20 residues, 6-15 residues, 6-10 residues, 7-30 residues, 7-25 residues, 7-20 residues, 7-15 residues, 7-10 residues, 8-30 residues, 8-25 residues, 8-20 residues, 8-15 residues, 8-10 residues, 9-30 residues, 9-25 residues, 9-20 residues, 9-15 residues, 9-10 residues, 10-30 residues, 10-25 residues, 10-20 residues, 10-15 residues, 10-12 residues, 11-30 residues, 11-25 residues, 11-20 residues, 11-15 residues, 12-30 residues, 12-25 residues, 12-20 residues, 12-15 residues, 13-30 residues, 13-25 residues, 13-20 residues, 13-15 residues, 14-30 residues, 14-25 residues, 14-20 residues, 14-15 residues, 15-30 residues, 15-25 residues, 15-20 residues, 16-30 residues, 16-25 residues, 16-20 residues, 17-30 residues, 17-25 residues, 17-20 residues, 18-30 residues, 18-25 residues, 18-20 residues, 19-30 residues, 19-25 residues, 19-20 residues, 20-30 residues, 20-25 residues, 20-22 residues, 21-30 residues, 21-25 residues, 22-30 residues, 22-25 residues, 23-30 residues, 23-25 residues, 24-30 residues, 24-25 residues, 24-27 residues, 25-30 residues, 25-28 residues, or 25-27 residues in length. In some embodiments, the peptide length may be at least 3, 4, 5, 6, 7, 8, 9, 10, 11, 12, 13, 14, 15, 16, 17, 18, 19, 20, 21, 22, 23, 24, 25, 26, 27, 28, 29, or 30 residues in length. In some embodiments, the peptide length may be no more than 30, 29, 28, 27, 26, 25, 24, 23, 22, 21, 20, 19, 18, 17, 16, 15, 14, 13, 12, 11, 10, 9, 8, 7, 6, 5, 4, or 3 residues.

In some aspects of the invention, the targeting agent and the active agent are covalently linked. In some embodiments, the targeting agent and the active agent are noncovalently linked.

In certain aspects of the invention, the targeting agent is characterized such that when it is contacted with a system comprising a targeted entity (e.g, a receptor on a cell in adipose tissue or a cancer cell.) and a targeting peptide that interacts specifically with the targeted entity, the targeting agent competes with the targeting peptide for interaction with the targeted entity.

In some embodiments, the invention provides a method of delivering a targeting agent to a targeted entity site that comprises delivering to the targeted entity site a targeting agent of the present invention.

In some aspects, the present invention provides a method of identifying a targeting agent comprising steps of providing a system comprising adipose or cancer cells or tissues and respectively non-adipose or non-cancer cells or tissues (the adipose cells may comprise prohibitin, and the cancer cells may comprise proteinase-3); providing a plurality of candidate targeting agents; contacting the system with members from the plurality; and determining that members of the plurality bind to and are internalized by the respective adipose or cancer cells, and not by the respective non-adipose or non-cancer cells in the system. The system may comprise an organism, such as a mammal, including a mouse, rat, or human. The targeting agents may be or may comprise polypeptides, including linear or cyclic polypeptides. The step of contacting may comprise contacting with expressed polypeptides, in some cases. In certain aspects, the polypeptides are displayed on viral particles.

In some embodiments, is the present invention provides a method comprising the step of identifying an agent that specifically binds to, and is internalized by, a receptor that is bound by a peptide selected from the group consisting of CKGGRAKDC (SEQ ID NO:1), CWELGGGPC (SEQ ID NO:2), CHVLGGGPC (SEQ ID NO:3), CVQGGGGPC (SEQ ID NO:4), and a combination thereof.

In certain embodiments, provided methods and compositions may be used to identify one or more receptors for a targeting peptide. In some embodiments, the compositions and methods may be used to identify naturally occurring ligands for known or newly identified receptors.

The present invention generally concerns ligand-receptor mapping by direct combinatorial selection in individuals with cancer.

In some embodiments, there is a pan-endothelial targeting agent characterized in that it interacts specifically with a targeted entity generally present on endothelial cells and is internalized into the cells. The targeted entity may be a vascular receptor or fragment thereof, and the targeted entity is or comprises prohibitin or is or comprises proteinase-3. In some embodiments, the targeting agent is or comprises a peptide, such as one comprising an amino acid sequence CKGGRAKDC (SEQ ID NO:1), CWELGGGPC (SEQ ID NO:2), CHVLGGGPC (SEQ ID NO:3), and/or CVQGGGGPC (SEQ ID NO:4). In some embodiments, the targeting agent comprises a peptide whose amino acid sequence shows 80%, 85%, 90%, 95%, 98%, 99%, or 100% overall identity with a portion of ANXA2 or RAGE that is at least 3 amino acids in length, and further includes an amino acid moiety as set forth in SEQ ID NOs: 1, 2, 3, or 4. wherein the pan-endothelial targeting agent is not ANXA2 or RAGE.

In some embodiments of a pan-endothelial targeting agent, the peptide is cyclic. In some embodiments, the targeted entity comprises an antibody or a fragment thereof. The pan-endothelial targeting agent may comprise a targeting agent linked with an active agent, such as one selected from the group consisting of a diagnostic agent and a therapeutic agent. In some embodiments, the active agent is or comprises a nucleic acid agent, such as DNA, RNA, siRNA, miRNA, antisense RNA or a combination thereof.

In some embodiments of a pan-endothelial targeting agent the active agent is or comprises an imaging agent, such as a radioisotope, for example one selected from the group consisting of ⁶⁴Cu, ¹¹¹In, ²¹³Bi, ¹⁰³Pd, ¹³³Xe, ¹³¹I, ⁶⁸Ge, ⁵⁷Co, ⁶⁵Zn, ⁸⁵Sr, ³²P, ³⁵S, ⁹⁰Y, ¹⁵³Sm, ¹⁵³Gd, ¹⁶⁹Yb, ⁵¹Cr, ⁵⁴Mn, ⁷⁵Se, ¹¹³Sn, ¹¹⁷Sn, ¹⁸⁶Re, ¹⁶⁶Ho and ¹⁸⁸Re, although in some embodiments the imaging agent is selected from the group consisting of an enzyme, a fluorescent label, a near infrared label, a luminescent label, a bioluminescent label, a magnetic label, and biotin.

In some embodiments, the active agent is or comprises an antibody agent, such as one selected from the group consisting of a monoclonal antibody, a polyclonal antibody, an Fc portion, an Fab, an ScFv, and a single domain antibody. In some embodiments, the active agent is or comprises a small molecule.

In some embodiments, the active agent is or comprises a peptide, such as a peptide of 3-30 residues in length, 5-25 residues in length, 8-20 residues in length, or 10-15 residues in length.

In some embodiments, the targeting agent and the active agent are covalently or are noncovalently linked.

The pan-endothelial targeting agent may be characterized in that, when it is contacted with a system comprising a targeted entity and a targeting peptide that interacts specifically with the targeted entity, the targeting agent competes with the targeting peptide for interaction with the targeted entity.

In some embodiments, there is a method of delivering a targeting agent to a targeted entity site, the method comprising step of delivering to the targeted entity site a targeting agent.

In some embodiments, there is a method of identifying a targeting agent, the method comprising steps of providing a system comprising adipose or cancer cells and non-adipose or cancer cells or tissues, respectively; providing a plurality of candidate targeting agents; contacting the system with members from the plurality; and determining that members of the plurality bind to and are internalized by the adipose or cancer cells, and not by non-adipose or non-cancer cells in the system. The system may comprise an organism, such as a human. In some embodiments, the targeting agents are or comprise polypeptides, for example polypeptides that are cyclic. The step of contacting may comprise contacting the system with expressed polypeptides (such as those 3-100 amino acids in length). In some embodiments, the polypeptides are displayed on viral particles. The cells or tissues may comprise prohibitin and/or proteinase-3.

In some embodiments, there is a method comprising the step of identifying an agent that specifically binds to, and is internalized by, a receptor that is bound by a peptide selected from the group consisting of CKGGRAKDC (SEQ ID NO:1), CWELGGGPC (SEQ ID NO:2), CHVLGGGPC (SEQ ID NO:3), and/or CVQGGGGPC (SEQ ID NO:4).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1. The schema represents a systematic approach used for the isolation of shared and tissue-specific ligand peptides in cancer patients. Step #1: three serial rounds of direct combinatorial selection were performed as indicated. Sequencing of DNA inserts encoding the displayed peptides provided the total number of peptides recovered in each round of selection. Step #2: Monte Carlo simulations and high-throughput tripeptide motif analyses were used to evaluate positive selection of peptides compared to the random peptide library. Step #3, shared and tissue-specific ligand peptide candidates were selected based on the analyses performed in Step #2. Step #4, biostatistical analysis is followed by ligand identification and receptor isolation, based on affinity chromatography, protein array screenings, or database mining. Steps #5, functional validation of the candidate ligand-receptor systems is performed at the protein, cell, and tissue levels.

FIG. 2. Combinatorial selection in patients. (A) Monte Carlo simulations with total number of ligand peptides analyzed in three consecutive rounds of selection show non-random distribution of tripeptide motifs. Thick black lines represent the Fisher's exact test; thin colored lines represent the corresponding random permutated dataset. (B) A saturation plot (modified from Dias-Neto et al., 2009) shows the number of distinct peptides as a function of the total number of peptide sequences obtained from DNA pyrosequencing and evaluated for each sampled tissue. All tissues reached saturation, as indicated by the “flattening” of the slope. In contrast, the unselected parental library showed no evidence of saturation. (C) Isolated peptides were grouped according to their tissue of origin and subjected to Monte Carlo simulation analysis. For every simulation, the pool of peptides was randomly distributed into groups corresponding to the number of sequences analyzed for each targeted tissue. Motif frequencies were calculated for each simulated organ, and Fisher's exact test was applied on the permutated dataset. A non-random selection of tripeptides was observed in all organs tested.

FIG. 3. Discovery of ANXA2/prohibitin as a tissue-specific ligand-receptor targeting normal human tissue. (A) Immunoblotting of His₆-ANXA2 or control proteins with rabbit antiserum against CKGGRAKDC-KLH or control pre-immune serum, as indicated. Arrow: His₆-ANXA2. (B) Binding of CKGGRAKDC-displaying phage is specifically inhibited by the corresponding synthetic peptide. Binding of unrelated control phage, insertless phage, binding to BSA and inhibition with an unrelated peptide served as controls. (C and D) Association of prohibitin and ANXA2 with membrane lipid rafts. Membrane proteins extracted from human WAT were subjected to immunoblotting or to fractionation enriching for non-caveolar or caveolar lipid rafts. Proteins recognized by anti-ANXA2 (C), anti-prohibitin (D, upper panel), and anti-caveolin 1 antibodies (D, lower panel) are indicated by arrows. (E) Binding of prohibitin and ANXA2 in vitro. Increasing concentrations of GST-prohibitin or GST tag control were captured with His₆-ANXA2 or control His₆-ANXAS. Specific binding was assessed with anti-GST antibodies. Arrow indicates GST-prohibitin (migrating as several bands). (F and G) Vascular expression of ANXA2 in human WAT Immunohistochemistry with anti-ANXA2 and anti-prohibitin antibodies on human WAT demonstrated co-localization of ANXA2 and prohibitin in the tissue vasculature. Blood vessels identity was confirmed by staining with anti-VE-cadherin antibody (G, inset). Arrows point to blood vessels. Red insets show lower magnification of the corresponding area. Scale bar, 100 p.m.

FIG. 4. Discovery of receptor for advanced glycation end-products (RAGE)/proteinase-3 (PR-3) as a ligand-binding pair targeting human bone marrow containing cancer cells. (A) Anti-CWELGGGPC antibodies recognize human recombinant RAGE in vitro. ELISA with post- and pre-immune rabbit polyclonal antibodies against CWELGGGPC (SEQ ID NO:2) was performed on immobilized KLH-conjugated CWELGGGPC, a control peptide, recombinant Fc-tagged proteins, and a control protein. (B) Anti-CWELGGGPC antibodies recognize native human RAGE. Protein extracts from human prostate cancer PC3 cells, DU145 cells, or from human bone marrow mononuclear control cells, along with recombinant RAGE-Fc protein, were immunoblotted with post- and pre-immune polyclonal antibodies against CWELGGGPC (SEQ ID NO:2) peptide. Arrow points to RAGE. (C) Validation of RAGE binding to PR-3 in vitro. Either immobilized PR-3 or control protein (BSA) was subjected to Fc-RAGE, Fc-BMPRIA, BSA, and anti-PR-3 antibody. Bars represent mean±s.e.m. (D) RAGE-Fc binding to active PR-3 is concentration-dependent. (E) Binding of CWELGGGPC-phage is specifically inhibited by the corresponding synthetic peptide. Binding of unrelated control phage, insertless phage, binding to BSA and inhibition with an unrelated peptide served as controls. (F) Relative quantification of RAGE expression on prostate cancer patient samples. Expression of RAGE is represented as low, moderate and high expression according to a standardized pathology scoring system. (G-I) Immunohistochemistry with RAGE-specific antibodies performed on human tissue sections derived from a panel of prostate cancer patients. (G) Organ-confined prostate cancer; (H), lymph node metastasis; and (I), bone marrow metastasis. Asterisks represent lymphoid (H) and bone (I) tissues. Scale bar, 100 p.m.

FIG. 5. (A) Left panel: peptides enriched in three rounds of selection in human WAT matched to the ANXA2 amino acid sequence. Underlining colors indicate the original round of selection. The CKGGRAKDC (SEQ ID NO:1) similarity sequence is highlighted in yellow. Right panel: peptides enriched in WAT matched to prohibitin. Similarity criteria: four or more amino acids identical (red) or conserved (blue) to the correspondingly positioned protein residues. (B) ANXA2 surface-exposed connector loops. Peptides enriched in WAT are shown.

FIG. 6. Co-localization of ANXA2 and prohibitin in the vasculature of WAT. Arrows point to blood vessels positively stained for ANXA2 (green) and prohibitin (red). DAPI (blue) indicates nuclear staining.

FIG. 7. Identification of the protein complex RAGE/leukocyte proteinase-3 as a molecular target of bone metastases. (A) Molecular modeling of the RAGE extracellular domain. Shown is the ribbon secondary structure conformation of RAGE. The surface-exposed WKLGGGP (SEQ ID NO:13) sequence is indicated (arrow). Similarity of bone marrow-homing peptide to the human receptor is shown. (B) Liquid chromatography-mass spectrometry (LC-MS/MS) of peptides matching the candidate receptor PR-3. Peptides identified are underlined. (Proteinase 3 sequence is SEQ ID NO:11.) (C) Isolation of PR-3 as a bone marrow target for the RAGE-like motif Monoavidin beads loaded with biotinylated synthetic peptides: a negative control peptide (sequence CARAC; indicated as control elution) or an equimolar admixture of CWELGGGPC (SEQ ID NO:2) plus CPGGGLVHC (SEQ ID NO: 6) (indicated as targeted elution), were incubated sequentially with a membrane protein extract from human white mononuclear bone marrow cells, washed, eluted by low pH, resolved by SDS-PAGE, and stained with Coomassie blue. Arrow: ˜35 kDa band specific to the RAGE-like peptides. (D) Similarity of human PR-3 to human EN-RAGE and human HMGB 1. Asterisks mark residues critical for RAGE binding to either HMGB 1 or EN-RAGE. Residues conserved between PR-3 and either HMGB1 or EN-RAGE are highlighted. Residues that are both critical for RAGE binding to HMGB1 or EN-RAGE and conserved between PR-3 and either HMGB1 or EN-RAGE are boxed. Amino acid residue color-coding: red, hydrophobic; green, neutral and polar; purple, basic; blue, acidic. The α-helix within the PR-3 C-terminus is underlined.

### DETAILED DESCRIPTION OF THE INVENTION

### I. Definitions

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

As used in this application, the terms “about” and “approximately” are used as equivalents. Any numerals used in this application with or without about/approximately are meant to cover any normal fluctuations appreciated by one of ordinary skill in the relevant art.

Antibody: As used herein, the term “antibody” is intended to include immunoglobulins and fragments thereof which are specifically reactive to the designated protein or peptide, or fragments thereof. Suitable antibodies include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), single chain antibodies, cameloid antibodies, antibody-like molecules, and antibody fragments. As used herein, the term “antibodies” also includes intact monoclonal antibodies, polyclonal antibodies, single domain antibodies (e.g., shark single domain antibodies (e.g., IgNAR or fragments thereof)), multispecific antibodies (e.g. bi-specific antibodies) formed from at least two intact antibodies, and antibody fragments so long as they exhibit the desired biological activity. Antibody polypeptides for use herein may be of any type (e.g., IgA, IgD, IgE, IgG, IgM).

Antibody fragment: As used herein, an “antibody fragment” includes a portion of an intact antibody, such as, for example, the antigen-binding or variable region of an antibody. Examples of antibody fragments include Fab, Fab′, F(ab′)2, Fc and Fv fragments; triabodies; tetrabodies; linear antibodies; single-chain antibody molecules; and multi specific antibodies formed from antibody fragments. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“ScFv proteins”), and minimal recognition units consisting of the amino acid residues that mimic the hypervariable region.

Characteristic Sequence Element: As used herein, the phrase a “characteristic sequence element” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, at least 10, at least 15, at least 20 or more amino acids are a characteristic of a protein. In general, a characteristic sequence element is one that, in addition to the sequence identity specified herein, shares at least one functional characteristic (e.g., biological activity, epitope, etc) with the relevant intact protein. In many embodiments, a characteristic sequence element is one that is present in all members of a family of polypeptides, and can be used to define such members.

Combination therapy: The term “combination therapy”, as used herein, refers to those situations in which two or more different pharmaceutical agents are administered in overlapping regimens so that the subject is simultaneously exposed to both agents.

Determine: Many methodologies described herein include a step of “determining”. Those of ordinary skill in the art, reading the present specification, will appreciate that such “determining” can utilize any of a variety of techniques available to those skilled in the art, including for example specific techniques explicitly referred to herein. In some embodiments, a determination involves manipulation of a physical sample. In some embodiments, a determination involves consideration and/or manipulation of data or information, for example utilizing a computer or other processing unit adapted to perform a relevant analysis. In some embodiments, a determination involves receiving relevant information and/or materials from a source.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regiment, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses.

Host: The term “host” is used herein to refer to a system (e.g., a cell, organism, etc.) in which a nucleic acid or polypeptide of interest is present. In some embodiments, a host is a system that expresses a particular polypeptide of interest.

Isolated: The term “isolated”, as used herein, refers to an agent or entity that has either (i) been separated from at least some of the components with which it was associated when initially produced (whether in nature or in an experimental setting); or (ii) produced by the hand of man. Isolated agents or entities may be separated from at least about 10%, at least about 20%, at least about 30%, at least about 40%, at least about 50%, at least about 60%, at least about 70%, at least about 80%, at least about 90%, or more of the other components with which they were initially associated. In some embodiments, isolated agents are more than 90%, 91%, 92%, 93%, 94%, 95%, 96%, 97%, 98%, 99% pure.

Nucleic acid molecule: The term “nucleic acid molecule” is used broadly to mean any polymer of two or more nucleotides, which are linked by a covalent bond such as a phosphodiester bond, a thioester bond, or any of various other bonds known in the art as useful and effective for linking nucleotides. Such nucleic acid molecules can be linear, circular or supercoiled, and can be single stranded or double stranded, e.g. single stranded or double stranded DNA, RNA or DNA/RNA hybrid. In some embodiments, nucleic acid molecules are or include nucleic acid analogs that are less susceptible to degradation by nucleases than are DNA and/or RNA. For example, RNA molecules containing 2′-O-methylpurine substitutions on the ribose residues and short phosphorothioate caps at the 3′- and 5′-ends exhibit enhanced resistance to nucleases (Green et al., Chem. Biol., 2:683-695 (1995), which is incorporated herein by reference). Similarly, RNA containing 2′-amino-2′-deoxypyrimidines or 2′-fluro-2′-deoxypyrimidines is less susceptible to nuclease activity (Pagratis et al., Nature Biotechnol., 15:68-73 (1997), which is incorporated herein by reference). Furthermore, L-RNA, which is a stereoisomer of naturally occurring D-RNA, is resistant to nuclease activity (Nolte et al., Nature Biotechnol., 14:1116-1119 (1996); Klobmann et al., Nature Biotechnol., 14:1112-1115 (1996); each of which is incorporated herein by reference). Such RNA molecules and methods of producing them are well known in the art and can be considered to be routine (see Eaton and Piekern, Ann. Rev. Biochem., 64:837-863 (1995), which is incorporated herein by reference). DNA molecules containing phosphorothioate linked oligodeoxynucleotides are nuclease resistant (Reed et al., Cancer Res. 50:6565-6570 (1990), which is incorporated herein by reference). Phosphorothioate-3′ hydroxypropylamine modification of the phosphodiester bond also reduces the susceptibility of a DNA molecule to nuclease degradation (see Tam et al., Nucl. Acids Res., 22:977-986 (1994), which is incorporated herein by reference).

Organ or Tissue: As used herein, the terms “organ or tissue” and “selected organ or tissue” are used in the broadest sense to mean an organ or tissue in or from a body. In some embodiments, an organ or tissue has a pathology, for example, tissue containing tumors (including lung containing tumors), whether primary or metastatic lesions. In some embodiments, an organ or tissue is normal (e.g., healthy). The term “control organ or tissue” is used to mean an organ or tissue other than a selected organ or tissue of interest. In some embodiments, a control organ or tissue is characterized by the inability of a ligand-encoding phage to home to the control organ or tissue and, therefore, is useful for identifying selective binding of a molecule to a selected organ or tissue.

Polypeptide: A “polypeptide”, generally speaking, is a string of at least two amino acids attached to one another by a peptide bond. In some embodiments, a polypeptide includes at least 3-5 amino acids, each of which is attached to others by way of at least one peptide bond. Those of ordinary skill in the art will appreciate that, in some embodiments, polypeptides include one or more “non-natural” amino acids or other entities that nonetheless are capable of integrating into a polypeptide chain. In some embodiments, a polypeptide may comprise, but is not limited to, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater amino acid residues. A polypeptide as described herein may be a member of a polypeptide family or class. As will be understood by those skilled in the art, polypeptide families or classes are defined by shared structural elements (e.g., preservation of one or more characteristic sequence elements, which may include sets of identical or similar residues separated from one another by defined distances, and/or a specified degree of overall sequence identity. In some embodiments, members of a polypeptide family or class share an overall sequence identity of at least about 40%, at least about 50%, at least about 60%, at least about 70%, at least about 80%, at least about 90%, at least about 90%, at least about 91%, at least about 92%, at least about 93%, at least about 94%, at least about 95%, at least about 96%, at least about 97%, at least about 98%, at least about 99% or more. In some embodiments, members of a polypeptide family or class shoe substantial sequence identity to one another. In some embodiments, members of a polypeptide family or class have similar lengths, typically not differing from each other by more than 50%, more than 45%, more than 40%, more than 35%, more than 30%, more than 25%, more than 20%, more than 15%, more than 10%, more than 5%, more than 4%, more than 3%, more than 2%, more than 1%, or less.

Predominantly present: The term “predominantly present”, as used herein to refer to amino acid residues in a polypeptide, refers to the presence of the residue at a particular location across a population. In some embodiments, an amino acid is considered to be predominantly present if, across a population of polypeptides, the particular amino acid is statistically present in at least about 50%, about 55%, about 60%, about 65%, about 70%, about 75%, about 80%, about 85%, about 90%, about 95%, about 96%, about 97%, about 98%, about 99% or more of the polypeptides.

Receptor: The term “receptor” for a targeting peptide includes but is not limited to any molecule or complex of molecules that binds to a targeting peptide. Non-limiting examples of receptors include peptides, proteins, glycoproteins, lipoproteins, epitopes, lipids, carbohydrates, multi-molecular structures, a specific conformation of one or more molecules and/or a morphoanatomic entity. In some embodiments, a “receptor” is a naturally occurring molecule or complex of molecules that is present on the surface of endothelial cells and/or endothelial tissue.

Sample: As used herein, the term “sample” refers to a cell, tissue, organ or portion thereof that is isolated from a body. It will be appreciated that a sample may be or comprise a single cell or a plurality of cells. In some embodiments, a sample is or comprises a histologic section or a specimen obtained by biopsy (e.g., surgical biopsy); in some embodiments, a sample is or comprises cells that are or have been placed in or adapted to tissue culture. In some embodiments, a sample is a specimen obtained from a dead body (e.g., by autopsy). In some embodiments, the sample is or comprises an intact organ or tissue. In some embodiments, the sample is or comprises circulating cells, such as circulating tumor cells.

Sample processing: As used herein, the term “sample processing” generally refers to various steps that may be accomplished to prepare a sample for quantification. In some embodiments, crude sample (e.g., whole tissue, homogenized tissue, etc.) is prepared. In some embodiments, purified or highly purified sample is prepared.

Specificity: As is known in the art, “specificity” is a measure of the ability of a particular ligand (e.g., a targeting peptide) to distinguish its binding partner (e.g., a target tissue, or organ of interest) from other potential binding partners (e.g., a control tissue or organ).

Subject: As used herein, the terms “subject,” “individual” or “patient” refer to a human or a non-human mammalian subject. In some embodiments, a subject is a non-human primate. In some embodiments, the subject is a dog, cat, goat, horse, pig, mouse, rabbit, or the like. In some embodiments, a subject is a human. In some embodiments, a subject is healthy. In some embodiments, a subject is suffering from or susceptible to a disease, disorder or condition (e.g., associated with the endothelium). In some embodiments, a human subject is a patient having a surgical tumor resection or a surgical biopsy. In some embodiments, a human subject is overweight, obese, has a metabolic condition related to being overweight or obese, or has cancer, is suspected of having cancer, or is at risk for developing cancer.

Substantial homology: The phrase “substantial homology” is used herein to refer to a comparison between amino acid or nucleic acid sequences. As will be appreciated by those of ordinary skill in the art, two sequences are generally considered to be “substantially homologous” if they contain homologous residues in corresponding positions. Homologous residues may be identical residues. In some embodiments, homologous residues may be non-identical residues that share one or more structural and/or functional characteristics. For example, as is well known by those of ordinary skill in the art, certain amino acids are typically classified as “hydrophobic” or “hydrophilic” amino acids, and/or as having “polar” or “non-polar” side chains In some embodiments, substitution of one amino acid for another of the same type is considered a “homologous” substitution. Typical amino acid categorizations are summarized below:

| | | | | | |
|---|---|---|---|---|---|
| Alanine | Ala | A | nonpolar | neutral | 1.8 |
| Arginine | Arg | R | polar | positive | −4.5 |
| Asparagine | Asn | N | polar | neutral | −3.5 |
| Aspartic | Asp | D | polar | negative | −3.5 |
| acid | | | | | |
| Cysteine | Cys | C | nonpolar | neutral | 2.5 |
| Glutamic | Glu | E | polar | negative | −3.5 |
| acid | | | | | |
| Glutamine | Gln | Q | polar | neutral | −3.5 |
| Glycine | Gly | G | nonpolar | neutral | −0.4 |
| Histidine | His | H | polar | positive | −3.2 |
| Isoleucine | Ile | I | nonpolar | neutral | 4.5 |
| Leucine | Leu | L | nonpolar | neutral | 3.8 |
| Lysine | Lys | K | polar | positive | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying homologous sequences, the programs mentioned above typically provide an indication of the degree of homology. In some embodiments, two sequences are considered to be substantially homologous if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are homologous over a relevant stretch of residues. In some embodiments, the relevant stretch is a complete sequence. In some embodiments, the relevant stretch is at least 6, at least 7, at least 8, at least 9, at least 10, at least 12, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul, et al., “Gapped BLAST and PSI-BLAST: a new generation of protein database search programs”, Nucleic Acids Res. 25:3389-3402, 1997; Baxevanis, et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999; all of the foregoing of which are incorporated herein by reference. In addition to identifying identical sequences, the programs mentioned above typically provide an indication of the degree of identity. In some embodiments, two sequences are considered to be substantially identical if at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or more of their corresponding residues are identical over a relevant stretch of residues. In some embodiments, the relevant stretch is a complete sequence. In some embodiments, the relevant stretch is at least 6, at least 7, at least 8, at least 9, at least 10, at least 12, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 225, at least 250, at least 275, at least 300, at least 325, at least 350, at least 375, at least 400, at least 425, at least 450, at least 475, at least 500 or more residues. In particular cases, a specific targeting peptide is described in the context of being substantially identical to another targeting peptide.

Therapeutic agent: As used herein, the phrase “therapeutic agent” refers to any agent that elicits a desired biological or pharmacological effect.

Treatment: As used herein, the term “treatment” refers to any method used to alleviate, delay onset, reduce severity or incidence, or yield prophylaxis of one or more symptoms or aspects of a disease, disorder, or condition. For the purposes of the present invention, treatment can be administered before, during, and/or after the onset of symptoms.

Unit dose form: As used herein, the term “unit dose form” refers to a physically discrete unit of a therapeutic agent for treatment of a patient. Each unit contains a predetermined quantity of active material calculated to produce the desired effect. It will be understood, however, that the total dosage of the composition will be decided by the attending physician within the scope of sound medical judgment

### II. General Embodiments of the Invention

Molecules differentially expressed in certain cells and/or tissues (including, for example, adipose cells or cancer cells) are attractive targets for therapy. Their direct identification within the vast human vasculature is challenging, although the present invention provides effective methods and compositions useful for their identification. The present invention generally concerns one or more agents that have the ability to target endothelial cells and endothelial tissues. In some embodiments, a targeting agent (e.g., an agent that targets adipose or cancer cells/tissues) comprises at least one peptide. In some embodiments, a targeting agent is linked with an active agent (e.g., a therapeutic agent, a diagnostic agent, or an imaging agent, among others).

In some embodiments, the provided targeting agents bind one or more markers, including one or more cell receptors. In some embodiments, targeted entities (e.g., ligand-receptors) are in common or specific to certain tissues. A distribution of certain ligand-receptor pairs was identified, including those having a restricted and specific distribution in normal tissue (prohibitin/annexin A2 in white adipose tissue) or cancer (RAGE/leukocyte proteinase-3 in bone metastases), for example. Thus, the present invention encompasses the recognition that these ligand/receptor pairs are useful for a variety of biotechnology and medical applications.

In aspects of the invention, CKGGRAKDC (SEQ ID NO:1) mimics annexin A2, and both compounds bind prohibitin, and these interactions are included in methods and compositions of the invention. Furthermore, CWELGGGPC (SEQ ID NO:2), CHVLGGPC (SEQ ID NO:3), and CVQGGGGPC (SEQ ID NO:4) are able to mimic RAGE and are able to bind proteinase-3. Particular tissues that may be targeted by CWELGGGPC (SEQ ID NO:2), CHVLGGPC (SEQ ID NO:3), and/or CVQGGGGPC (SEQ ID NO:4) include, for example, primary bone cancer, including cancer of the bone marrow and cancers that have metastasized to bone, including from prostate, for example.

### III. Cell Markers

Cell markers in accordance with the present invention include entities that are targeted by targeting agents of the invention. It will be appreciated that the markers may be of any kind of gene product to which a targeting agent is able to bind. In some embodiments, a targeting agent is internalized upon binding to the marker. A marker may be considered a celltarget site (a region on the surface of a cell that may or may not be proteinaceous in nature, such as lipid or carbohydrate, for example), cell receptor (including the entire receptor or the extracellular domain, for example), cell receptor peptide (for example, a peptide fragment identical to or substantially identical to part of a receptor protein), or receptor entity. In some embodiments, the targets are or comprise receptors in the cell surface membranes of cells, including of adipose or cancer cells. Receptors may be of any kind of cell receptors targeted by one or more targeting agents. In some embodiments, particular receptors are targeted in accordance with the invention. Non-limiting marker receptors include, e.g., prohibitin or proteinase-3 for white adipose tissue (including adipocytes) and cancer, respectively.

In some embodiments of the invention, targeted agents are internalized into targeted cells (e.g., upon or after binding). Methods of identifying and/or quantitating internalization of the targeted agent upon binding to the marker are known in the art (see, for example, Henrizues et al., 2007 for review). In some embodiments, methods for identifying and/or quantitating internalization employ mass spectrometry (Jiao et al., 2009), fluorescence and flow cytometry (Benincasa et al., 2009), fluorescence and confocal microscopy (Nakase et al., 2004), and biotinylation and phase contrast imaging (Cardo-Vila et al., 2003), for example.

### IV. Targeting Agents

In some embodiments of the invention, there are targeting agents that are able to target a specific cellular marker. For example, in some embodiments, there are targeting agents that are able to target endothelial cells and in particular cases become internalized therein. Provided targeting agents may target adipose tissue or cancer. The targeting agent may be of any kind, but in some embodiments, the targeting agent comprises a small molecule or peptide. In some embodiments of the invention, a peptide has the general formula of CX₇C (SEQ ID NO:12) and may be cyclical. In some cases the peptide is about nine amino acids in length. In some cases, the peptide is identified with the entire motif of SEQ ID NO:12. In some embodiments, a peptide is employed without the N-terminal and C-terminal cysteines.

In some embodiments, peptides direct one or more compositions to cells or tissues. Provided compositions may be of any kind so long as they are able to be combined or attached to a targeting agent (including a peptide). In some embodiments, a composition is an active agent, such as a therapeutic agent and/or an imaging agent. The therapeutic agent may be of any kind, including but not limited to, e.g., a drug, radioisotope, killer peptide, antibody, RNAi molecule (including siRNA or miRNA), vectors (including gene therapy vectors), fusion proteins, and so forth.

In some embodiments, an active agent is a diagnostic agent, including, e.g., an imaging agent, such as radioisotopes, enzymes, fluorescent label, luminescent label, bioluminescent label, magnetic label, biotin, etc. In some embodiments of the invention, the targeting peptide/imaging agent composition is localized in vivo, and such information is useful to a medical provider, for example to assist in diagnosis of a medical condition in the individual. Imaging of the composition may identify location of the composition within certain cells and tissues (e.g., adipose cells and tissues or cancer cells). The imaging may assist in a diagnosis of a disease or may be definitive for diagnosis of a disease. The imaging may be utilized in conjunction with other medical information (such as Ankle Brachial Index, computerized tomography (CT) scan, ultrasound Doppler test, magnetic resonance angiography, biopsy, blood test, and/or stress test, for example) to determine a diagnosis for a subject.

In some embodiments, a targeting peptide is utilized with a compound that is both therapeutic and traceable, such as a radioisotope.

In some embodiments, an active agent may be physically bound to the peptide so long as such binding does not interfere with the function of the peptide to localize to its target or with the function of the active agent. In some cases, the peptide may be covalently or noncovalently bound to the active agent (e.g., therapeutic or imaging agent). In some embodiments, the peptide is conjugated to the active agent. The active agent may be bound directly or indirectly to the peptide. Exemplary active agents include nucleic acids, antibody agents, peptides, small molecules, and so forth. The active agent may be a drug, such as an endothelial disease drug or an anti-cancer drug.

Particular targeting peptides of the invention include those comprising particular sequences or variants thereof. In some embodiments, targeting peptides include one or more of amino acid sequences, CWELGGGPC (SEQ ID NO:2), CHVLGGGPC (SEQ ID NO:3), and/or CVQGGGGPC (SEQ ID NO:4), or variants thereof. In some embodiments, variants retain the function to target markers and to be internalized. Aspects of the peptides include those having conservative or non-conservative amino acid substitutions, for example at one, two, three, four, five, or more amino acids of the peptide. In some aspects, provided peptides are at least 70%, 75%, 80%, 85%, 90%, 95%, or more identical to SEQ ID NOS:1, 2, 3, or 4, for example. In some aspects, provided peptides are between 70% and 99% identical, between 70% and 95% identical, between 70% and 90% identical, between 70% and 85% identical, between 70% and 80%, between 70% and 75% identical, between 75% and 99% identical, between 75% and 95% identical, between 75% and 90% identical, between 75% and 85% identical, between 75% and 80%, between 80% and 99% identical, between 80% and 95% identical, between 80% and 90% identical, between 80% and 85% identical, between 85% and 99% identical, between 85% and 95% identical, between 85% and 90% identical, between 90% and 99% identical, or between 90% and 95% identical to SEQ ID NOS:1, 2, 3, or 4, for example.

In certain embodiments, the present invention concerns novel compositions comprising at least one targeting peptide. As used herein, a peptide generally refers, but is not limited to a peptide of from about 3 to about 100 amino acids, or from about 3 to about 75, or about 3 from about 60, or from about 3 to about 50, or from about 3 to about 40, or from about 3 to about 30, or from about 3 to about 25, or from about 3 to about 20, or from about 3 to about 10, or from about 3 to about 9, or from about 5 to about 25, or from about 8 to about 20, or from about 10 to 15 in length.

In certain embodiments the size of the at least one peptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 or more amino acid residues.

As used herein, an “amino acid residue” refers to any naturally occurring amino acid, any amino acid derivative or any amino acid mimic known in the art. In some embodiments, the residues of the protein or peptide are sequential, without any non-amino acid interrupting the sequence of amino acid residues. In some embodiments, the sequence may comprise one or more non-amino acid moieties. In some embodiments, the sequence of residues of the protein or peptide may be interrupted by one or more non-amino acid moieties.

As used herein, the term “peptide” encompasses amino acid sequences comprising at least one of the 20 common amino acids found in naturally occurring proteins, or at least one modified or unusual amino acid.

Peptides may be made by any technique known to those of skill in the art, including the expression of peptides through standard molecular biological techniques, the isolation of peptides from natural sources, or the chemical synthesis of peptides. In some embodiments, peptides are synthesized chemically. In some cases, the nucleotide and peptide sequences corresponding to various genes or gene fragments may be found at computerized databases known to those of ordinary skill in the art. One such database is the National Center for Biotechnology Information's GenBank® and GenPept databases. The coding regions for known genes may be amplified and/or expressed using the techniques disclosed herein or as would be known to those of ordinary skill in the art. Various commercial preparations of peptides are known to those of skill in the art.

Modifications and/or changes may be made in the structure of polynucleotides and and/or peptides according to the present invention, while obtaining molecules having similar or improved characteristics, such biologically functional equivalents are also encompassed within the present invention. In some such embodiments, targeting peptides retain the ability to target be internalized in an endothelial cell.

The biological functional equivalent may comprise a polynucleotide that has been engineered to contain distinct sequences while at the same time retaining the capacity to encode a particular targeting peptide. This can be accomplished to the degeneracy of the genetic code, i.e., the presence of multiple codons, which encode for the same amino acids. In one example, one of skill in the art may wish to introduce a restriction enzyme recognition sequence into a polynucleotide while not disturbing the ability of that polynucleotide to encode a protein.

In another example, a polynucleotide made be (and encode) a biological functional equivalent with more significant changes. Certain amino acids may be substituted for other amino acids in a peptide structure without appreciable loss of interactive binding capacity with structures such as, for example, antigen-binding regions of antibodies, binding sites on substrate molecules, receptors, and such like. So-called “conservative” changes do not disrupt the biological activity of the protein, as the structural change is not one that impinges of the peptide's ability to carry out its designed function. It is thus contemplated by the inventors that various changes may be made in the sequence of genes and peptides disclosed herein, while still fulfilling the goals of the present invention.

In terms of functional equivalents, it is well understood by the skilled artisan that, inherent in the definition of a “biologically functional equivalent” peptide, is the concept that there is a limit to the number of changes that may be made within a defined portion of the molecule while retaining a molecule with an acceptable level of equivalent biological activity. Biologically functional equivalents are thus defined herein as those peptides (and polynucleotides) in selected amino acids (or codons) may be substituted. Functional activity includes the ability to target an active agent to a particular vascular bed, including certain endothelial cells.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (0.4); threonine (0.7); serine (0.8); tryptophan (0.9); tyrosine (1.3); proline (1.6); histidine (3.2); glutamate (3.5); glutamine (3.5); aspartate (3.5); asparagine (3.5); lysine (3.9); and/or arginine (4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is useful, those which are within ±1 are particularly useful, and/or those within ±0.5 are even more particularly useful.

It also is understood in the art that the substitution of like amino acids can be made effectively on the basis of hydrophilicity, particularly where the biological functional equivalent peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (0.4); proline (−0.5±1); alanine (0.5); histidine (0.5); cysteine (1.0); methionine (1.3); valine (1.5); leucine (1.8); isoleucine (1.8); tyrosine (2.3); phenylalanine (2.5); tryptophan (3.4). In making changes based upon similar hydrophilicity values, the substitution of amino acids whose hydrophilicity values are within ±2 is useful, those which are within ±1 are particularly useful, and/or those within ±0.5 are even more particularly useful.

A. Peptide Mimetics

Peptide mimetics may be used in accordance with the present invention. In general, peptide mimetics are peptide-containing molecules that mimic elements of protein secondary structure. See, for example, Johnson et al., “Peptide Turn Mimetics” in BIOTECHNOLOGY AND PHARMACY, Pezzuto et al., Eds., Chapman and Hall, New York (1993), incorporated herein by reference. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used to engineer second generation molecules having many of the natural properties of the targeting peptides disclosed herein, but with altered and even improved characteristics.

B. Peptide Purification

In certain embodiments a peptide may be isolated or purified. Peptide purification techniques are well known to those of skill in the art. These techniques involve, at one level, the homogenization and crude fractionation of the cells, tissue or organ to polypeptide and non-polypeptide fractions. The protein or polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. An example of receptor protein purification by affinity chromatography is disclosed in U.S. Pat. No. 5,206,347, the entire text of which is incorporated herein by reference. A particularly efficient method of purifying peptides is fast protein liquid chromatography (FPLC) or even HPLC.

A purified protein or peptide is intended to refer to a composition, isolatable from other components, wherein the protein or peptide is purified to any degree relative to its naturally-obtainable state. An isolated or purified protein or peptide, therefore, also refers to a protein or peptide free from the environment in which it may naturally occur. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide are known to those of skill in the art in light of the present disclosure. These include, for example, determining the specific activity of an active fraction, or assessing the amount of polypeptides within a fraction by SDS/PAGE analysis. A method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification are well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance to be isolated and a molecule to which it can specifically bind to. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.). The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand.

C. Synthetic Peptides

Because of their relatively small size, targeting peptides of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, usually from about 6 up to about 35 to 50 amino acids, can be readily synthesized by such methods. In some embodiments, recombinant DNA technology may be employed wherein a nucleotide sequence which encodes a peptide of the invention is inserted into an expression vector, transformed or transfected into an appropriate host cell, and cultivated under conditions suitable for expression.

### V. Identification and/or Characterization of Targeting Agents

The present invention includes the identification and/or characterization of targeting agents. In certain aspects, there are methods that include contacting a system with one or more candidate targeting peptides and assaying for internalization of the candidate peptide.

In some embodiments, there are methods that include providing a system having certain cells and other cells or tissues, providing a plurality of candidate targeting agents, contacting the system with members from the plurality, and determining that members of the plurality bind to and are internalized by the certain cells, and not by the other cells in the system.

It will be appreciated that one can identify adipose or cancer targeting agents by providing a library of candidate peptides to a human (such as, for example, a phage display library), biopsying particular tissue from the individual, employing an algorithm to perform Monte Carlo simulation, and high throughput analysis utilizing similarity searching, protein arrays, and/or affinity chromatography to characterize the candidate targeting peptide (see Examples section).

In some embodiments of the invention, a “targeting peptide” is a peptide comprising a contiguous sequence of amino acids, that is characterized by selective localization to certain tissues or cells. Selective localization may be determined, for example, by methods disclosed herein, wherein the candidate targeting peptide sequence is displayed on the outer surface of a phage. Administration to a subject of a library of such phage that have been genetically engineered to express a multitude of such targeting peptides of different amino acid sequence is followed by collection of one or more tissues or cells from the subject and identification of phage found in that tissue or cells. A phage expressing a targeting peptide sequence is considered to be selectively localized to certain tissue or cells if it exhibits greater binding in that tissue or cells compared to a control tissue or organ. In some embodiments, selective localization of a targeting peptide should result in two-fold or higher enrichment of the phage in the target endothelial tissue or endothelial cells compared to a control organ, tissue or cell type. Selective localization resulting in at least a three-fold, four-fold, five-fold, six-fold, seven-fold, eight-fold, nine-fold, ten-fold or higher enrichment in the target organ compared to a control organ, tissue or cell type is useful in the invention. In some embodiments, a phage expressing a targeting peptide sequence that exhibits selective localization shows an increased enrichment in the target tissue or cells compared to a control organ when phage recovered from the target tissue or cells are reinjected into a second host for another round of screening. Further enrichment may be exhibited following a third or more round of screening, for example. In some embodiments to determine selective localization, phage expressing the candidate target peptide exhibits a two-fold or a three-fold or higher enrichment in the target tissue or cells compared to control phage that express a non-specific peptide or that have not been genetically engineered to express any candidate target peptides. Another means to determine selective localization is that localization to the target tissue or cells of phage expressing the target peptide is at least partially blocked by the co-administration of a synthetic peptide containing the target peptide sequence.

### VI. Administration

In embodiments of the invention, the compositions of the invention are delivered to a subject in need thereof. In some embodiments, a subject is a mammal, for example non-human primate, human, dog, cat, horse, cow, pig, sheep, or goat. Targeting agents may be delivered to a subject known to have one or more markers of the invention or suspected of having one or more markers (for example, suspected because the individual has at least one symptom of a particular disease). In some cases, a first composition comprising a targeting agent linked directly or indirectly to an imaging agent is delivered to the subject. Following imaging of the first composition, a second composition that comprises a targeting agent linked directly or indirectly to a therapeutic agent is delivered to the subject; the targeting agent may be the same or a different in the first and second compositions.

Where clinical applications are contemplated, pharmaceutical compositions including at least the targeting agent (and in some cases also an active agent) are prepared in a form appropriate for the intended application, such as treatment or diagnosis of an endothelial disease. Generally, this may entail preparing compositions that are essentially free of impurities that could be harmful to humans or animals, for example.

One generally will desire to employ appropriate salts and buffers to render delivery compositions stable and allow for uptake by target cells, where appropriate. Buffers may also be employed in the composition for introduction into a subject. Aqueous compositions of the present invention comprise an effective amount of the targeting peptide composition, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the targeting agents of the present invention, its use in therapeutic compositions is contemplated. Supplementary active ingredients also can be incorporated into the compositions.

Active compositions of the present invention may include classic pharmaceutical preparations. Administration of these compositions according to the present invention are via any common route so long as the target tissue is available via that route. This includes oral, nasal, buccal, rectal, vaginal or topical. In some embodiments, administration may be by orthotopic, intradermal, subcutaneous, intramuscular, intraperitoneal, intraarterial or intravenous injection. Such compositions normally would be administered as pharmaceutically acceptable compositions, described supra.

Pharmaceutical forms suitable for injectable use include sterile aqueous solutions or dispersions and sterile powders for the extemporaneous preparation of sterile injectable solutions or dispersions. In general, pharmaceutical forms are sterile and are fluid to the extent that easy syringability exists. It will generally be stable under the conditions of manufacture and storage and is preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the desired particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is useful to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in a desired amount in the appropriate solvent with various other ingredients enumerated above, followed by filtered sterilization. Generally, dispersions are prepared by incorporating the various sterilized active ingredients into a sterile vehicle which contains the basic dispersion medium and the other ingredients from those enumerated above. In some embodiments of using sterile powders for the preparation of sterile injectable solutions, the methods of preparation are vacuum-drying and freeze-drying techniques which yield a powder of the active ingredient plus any additional desired ingredient from a previously sterile-filtered solution thereof.

### VII. Active Agents

In aspects of the invention, a targeting agent is delivered with an active agent to a subject in need thereof, such as one that suffers from or is susceptible to a disease, disorder, or condition, including obesity or related conditions or cancer. An active agent may be of any kind including, but not limited to, a therapeutic agent and/or a diagnostic agent, e.g., an imaging agent. In some embodiments, an active agent is administered to healthy cells and/or tissues in an individual, including healthy endothelial cells and/or tissues.

A. Therapeutic Agents

Therapeutic agents may be of any kind so long as they are able to be delivered with a targeting agent of the invention. Therapeutic agents generally alleviate at least one symptom of a disease. One or more therapeutic agents may be employed with the active agent. A therapeutic agent may be combined with an active agent as a single composition. A therapeutic agent may be combined with a targeting agent in any manner so long as the targeting agent is able to localize the therapeutic agent to the target. A targeting agent and therapeutic agent may be utilized as a single entity wherein the two moieties are physically linked, such as through covalent binding.

1. Nucleic Acids

In some embodiments, the therapeutic agent comprises a nucleic acid, including DNA, RNA, RNAi (siRNA, miRNA), antisense RNA, and so forth. The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The term “nucleic acid” encompass the terms “oligonucleotide” and “polynucleotide,” each as a subgenus of the term “nucleic acid.” The term “oligonucleotide” refers to a molecule of between about 3 and about 100 nucleobases in length. The term “polynucleotide” refers to at least one molecule of greater than about 100 nucleobases in length. These definitions generally refer to a single-stranded molecule, but in some embodiments encompass an additional strand that is partially, substantially or fully complementary to the single-stranded molecule. Thus, a nucleic acid may encompass a double-stranded molecule or a triple-stranded molecule that comprises one or more complementary strand(s) or “complement(s)” of a particular sequence comprising a molecule.

a. Nucleobases

As used herein a “nucleobase” refers to a heterocyclic base, such as for example a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in manner that may substitute for naturally occurring nucleobase pairing (e.g., the hydrogen bonding between A and T, G and C, and A and U).

“Purine” and/or “pyrimidine” nucleobase(s) encompass naturally occurring purine and/or pyrimidine nucleobases and also derivative(s) and analog(s) thereof, including but not limited to, those a purine or pyrimidine substituted by one or more of an alkyl, caboxyalkyl, amino, hydroxyl, halogen (i.e., fluoro, chloro, bromo, or iodo), thiol or alkylthiol moeity. Preferred alkyl (e.g., alkyl, caboxyalkyl, etc.) moeities comprise of from about 1, about 2, about 3, about 4, about 5, to about 6 carbon atoms. Other non-limiting examples of a purine or pyrimidine include a deazapurine, a 2,6-diaminopurine, a 5-fluorouracil, a xanthine, a hypoxanthine, a 8-bromoguanine, a 8-chloroguanine, a bromothymine, a 8-aminoguanine, a 8-hydroxyguanine, a 8-methylguanine, a 8-thioguanine, an azaguanine, a 2-aminopurine, a 5-ethylcytosine, a 5-methylcyosine, a 5-bromouracil, a 5-ethyluracil, a 5-iodouracil, a 5-chlorouracil, a 5-propyluracil, a thiouracil, a 2-methyladenine, a methylthioadenine, a N,N-diemethyladenine, an azaadenines, a 8-bromoadenine, a 8-hydroxyadenine, a 6-hydroxyaminopurine, a 6-thiopurine, a 4-(6-aminohexyl/cytosine), and the like.

A nucleobase may be comprised in a nucleoside (an individual chemical unit comprising a nucleobase covalently attached to a nucleobase linker moiety) or nucleotide (a nucleoside further comprising a “backbone moiety”), using any chemical or natural synthesis method described herein or known to one of ordinary skill in the art. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar where a carbon is substituted for an oxygen atom in the sugar ring.

b. Nucleic Acid Analogs

A nucleic acid may comprise, or be composed entirely of, a derivative or analog of a nucleobase, a nucleobase linker moiety and/or backbone moiety that may be present in a naturally occurring nucleic acid. As used herein a “derivative” refers to a chemically modified or altered form of a naturally occurring molecule, while the terms “mimic” or “analog” refer to a molecule that may or may not structurally resemble a naturally occurring molecule or moiety, but possesses similar functions. As used herein, a “moiety” generally refers to a smaller chemical or molecular component of a larger chemical or molecular structure. Nucleobase, nucleoside and nucleotide analogs or derivatives are well known in the art, and have been described (see for example, Scheit, 1980, incorporated herein by reference). Additional non-limiting examples of nucleosides, nucleotides or nucleic acids comprising 5-carbon sugar and/or backbone moiety derivatives or analogs, include those in U.S. Pat. Nos. 5,681,947; 5,652,099; 5,763,167; 5,614,617; 5,670,663; 5,872,232; 5,859,221; 5,446,137; 5,886,165; 5,714,606; and 5,672,697, for example.

c. Polyether and Peptide Nucleic Acids

In certain embodiments, it is contemplated that a nucleic acid comprising a derivative or analog of a nucleoside or nucleotide may be used in the methods and compositions of the invention. A non-limiting example is a “polyether nucleic acid”, described in U.S. Pat. No. 5,908,845. In a polyether nucleic acid, one or more nucleobases are linked to chiral carbon atoms in a polyether backbone.

Another non-limiting example is a “peptide nucleic acid”, also known as a “PNA”, “peptide-based nucleic acid analog” or “PENAM”, described in U.S. Pat. Nos. 5,786,461, 5891,625, 5,773,571, 5,766,855, 5,736,336, 5,719,262, 5,714,331, 5,539,082. Peptide nucleic acids generally have enhanced sequence specificity, binding properties, and resistance to enzymatic degradation in comparison to molecules such as DNA and RNA. A peptide nucleic acid generally comprises one or more nucleotides or nucleosides that comprise a nucleobase moiety, a nucleobase linker moeity that is not a 5-carbon sugar, and/or a backbone moiety that is not a phosphate backbone moiety. Examples of nucleobase linker moieties described for PNAs include aza nitrogen atoms, amido and/or ureido tethers (see for example, U.S. Pat. No. 5,539,082). Examples of backbone moieties described for PNAs include an aminoethylglycine, polyamide, polyethyl, polythioamide, polysulfinamide or polysulfonamide backbone moiety.

d. RNA Interference

In some embodiments, the therapeutic agent comprises an RNA interfering molecule. The term “RNA interference” or “RNAi” refers generally to a process in which a double-stranded RNA molecule changes the expression of a nucleic acid sequence with which the double-stranded or short hairpin RNA molecule shares substantial or total homology. The term “RNAi agent” refers to an RNA sequence that elicits RNAi. The terms “short hairpin RNA” or “shRNA” refer to an RNA structure having a duplex region and a loop region. In some embodiments of the present invention, ddRNAi agents are expressed initially as shRNAs. In some embodiments, RNAi agents are expressed initially as shRNAs and comprise two or more stem-loop structures separated by one or more spacer region(s).

1) miRNA Molecules

MicroRNA molecules (“miRNAs”) are generally 21 to 22 nucleotides in length, though lengths of 19 and up to 23 nucleotides have been reported. The miRNAs are each processed from a longer precursor RNA molecule (“precursor miRNA”). Precursor miRNAs are transcribed from non-protem-encoding genes. The precursor miRNAs have two regions of complementarity that enable them to form a stem-loop- or fold-back-like structure, which is cleaved by an enzyme called Dicer in animals. Dicer is a ribonuclease III-like nuclease. The processed miRNA is typically a portion of the stem. The processed miRNA (also referred to as “mature miRNA”) become part of a large complex to down-regulate a particular target gene. Examples of animal miRNAs include those that imperfectly basepair with the target, which halts translation (Olsen et al, 1999; Seggerson et al, 2002) SiRNA molecules also are processed by Dicer, but from a long, double-stranded RNA molecule. SiRNAs are not naturally found in animal cells, but they can function in such cells in a RNA-induced silencing complex (RISC) to direct the sequence-specific cleavage of an mRNA target (Denli et al., 2003).

2) Small Interfering RNA (siRNA)

In some embodiments, siRNA is employed as the therapeutic agent. By “small interfering RNA” is meant a nucleic acid molecule which has complementarity in a substrate binding region to a specified gene target, and which acts to specifically guide enzymes in the host cell to cleave the target RNA. That is, the small interfering RNA by virtue of the specificity of its sequence and its homology to the RNA target, is able to cause cleavage of the RNA strand and thereby inactivate a target RNA molecule because it is no longer able to be transcribed. These complementary regions allow sufficient hybridization of the small interfering RNA to the target RNA and thus permit cleavage. One hundred percent complementarity is often useful for biological activity, but complementarity as low as 90% may be employed, for example.

In some embodiments, small interfering RNAs are double stranded RNA agents that have complementary to (i.e., able to base-pair with) a portion of the target RNA (generally messenger RNA). Generally, such complementarity is 100%, but can be less if desired, such as 91%, 92%, 93%, 94%, 95%, 96%, 97%, 98%, or 99%. For example, 19 bases out of 21 bases may be base-paired. In some instances, where selection between various allelic variants is desired, 100% complementary to the target gene is useful in order to effectively discern the target sequence from the other allelic sequence. When selecting between allelic targets, choice of length is also an important factor because it is the other factor involved in the percent complementary and the ability to differentiate between allelic differences.

The small interfering RNA sequence needs to be of sufficient length to bring the small interfering RNA and target RNA together through complementary base-pairing interactions. The small interfering RNA of the invention may be of varying lengths. The length of the small interfering RNA is preferably greater than or equal to ten nucleotides and of sufficient length to stably interact with the target RNA; specifically 15-30 nucleotides; more specifically any integer between 15 and 30 nucleotides, such as 15, 16, 17, 18, 19, 20, 21, 22, 23, 24, 25, 26, 27, 28, 29, and 30. By “sufficient length” is meant an oligonucleotide of greater than or equal to 15 nucleotides that is of a length great enough to provide the intended function under the expected condition. By “stably interact” is meant interaction of the small interfering RNA with target nucleic acid (e.g., by forming hydrogen bonds with complementary nucleotides in the target under physiological conditions).

e. Antisense RNA

In some embodiments, antisense RNA is employed as the therapeutic agent. Antisense RNA comprises a single-stranded RNA that is complementary to another nucleic acid, such as a mRNA strand. Antisense RNA may be introduced into a cell to inhibit translation of a particular complementary mRNA by hybridizing to it and physically obstructing the translation machinery.

2. Antibodies

In some embodiments, it may be desirable to make antibodies against identified targeting peptides or their receptors. An appropriate targeting peptide or receptor, or portions thereof, may be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions are familiar to those of skill in the art and should be suitable for administration to humans, i.e., pharmaceutically acceptable. Exemplary carriers include, but are not limited to, keyhole limpet hemocyanin (KLH) and bovine serum albumin (BSA).

In general, the term “antibody” is used to refer to any antibody-like molecule that has an antigen binding region, and includes antibody fragments such as Fab′, Fab, F(ab′).sub.2, single domain antibodies (DABs), Fv, scFv (single chain Fv), and the like. Techniques for preparing and using various antibody-based constructs and fragments are well-known in the art. Means for preparing and characterizing antibodies are also well known in the art (See, e.g., Antibodies: A Laboratory Manual, Cold Spring Harbor Laboratory, 1988; incorporated herein by reference).

3. Cytokines and Chemokines

In some embodiments, it may be desirable to couple specific bioactive agents to one or more targeting peptides for targeted delivery to an organ, tissue or cell type. Such agents include, but are not limited to, cytokines, chemokines, pro-apoptosis factors and anti-angiogenic factors. The term “cytokine” is a generic term for proteins released by one cell population which act on another cell as intercellular mediators. Examples of such cytokines are lymphokines, monokines, growth factors and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; prostaglandin, fibroblast growth factor; prolactin; placental lactogen, OB protein; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-.beta.; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1α, IL-2, IL-3, IL-4, IL-5, IL-6, IL-7, IL-8, IL-9, IL-10, IL-11, IL-12; IL-13, IL-14, IL-15, IL-16, IL-17, IL-18, LIF, G-CSF, GM-CSF, M-CSF, EPO, kit-ligand or FLT-3, angiostatin, thrombospondin, endostatin, tumor necrosis factor and LT. As used herein, the term cytokine includes proteins from natural sources or from recombinant cell culture and biologically active equivalents of the native sequence cytokines.

Chemokines generally act as chemoattractants to recruit immune effector cells to the site of chemokine expression. It may be advantageous to express a particular chemokine gene in combination with, for example, a cytokine gene, to enhance the recruitment of other immune system components to the site of treatment. Chemokines include, but are not limited to, RANTES, MCAF, MIP1-alpha, MIP1-Beta, and IP-10. The skilled artisan will recognize that certain cytokines are also known to have chemoattractant effects and could also be classified under the term chemokines.

In certain embodiments, chemotherapeutic agents may be attached to a targeting peptide or fusion protein for selective delivery to a tumor. Agents or factors suitable for use may include any chemical compound that induces DNA damage when applied to a cell. Chemotherapeutic agents include, but are not limited to, 5-fluorouracil, bleomycin, busulfan, camptothecin, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raloxifene, tamoxifen, taxol, temazolomide (an aqueous form of DTIC), transplatinum, vinblastine and methotrexate, vincristine, or any analog or derivative variant of the foregoing. Most chemotherapeutic agents fall into the following categories: alkylating agents, antimetabolites, antitumor antibiotics, corticosteroid hormones, mitotic inhibitors, and nitrosoureas, hormone agents, miscellaneous agents, and any analog or derivative variant thereof.

Chemotherapeutic agents and methods of administration, dosages, etc. are well known to those of skill in the art (see for example, the “Physicians Desk Reference”, Goodman & Gilman's “The Pharmacological Basis of Therapeutics” and in “Remington's Pharmaceutical Sciences”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject. Examples of specific chemotherapeutic agents and dose regimes are also described herein. Of course, all of these dosages and agents described herein are exemplary rather than limiting, and other doses or agents may be used by a skilled artisan for a specific patient or application. Any dosage in-between these points, or range derivable therein is also expected to be of use in the invention.

4. Alkylating Agents

Alkylating agents are drugs that directly interact with genomic DNA to prevent the cancer cell from proliferating. This category of chemotherapeutic drugs represents agents that affect all phases of the cell cycle, that is, they are not phase-specific. An alkylating agent, may include, but is not limited to, a nitrogen mustard, an ethylenimene, a methylmelamine, an alkyl sulfonate, a nitrosourea or a triazines. They include but are not limited to: busulfan, chlorambucil, cisplatin, cyclophosphamide (cytoxan), dacarbazine, ifosfamide, mechlorethamine (mustargen), and melphalan.

5. Antimetabolites

Antimetabolites disrupt DNA and RNA synthesis. Unlike alkylating agents, they specifically influence the cell cycle during S phase. Antimetabolites can be differentiated into various categories, such as folic acid analogs, pyrimidine analogs and purine analogs and related inhibitory compounds. Antimetabolites include but are not limited to, 5-fluorouracil (5-FU), cytarabine (Ara-C), fludarabine, gemcitabine, and methotrexate.

6. Natural Products

Natural products generally refer to compounds originally isolated from a natural source, and identified has having a pharmacological activity. Such compounds, analogs and derivatives thereof may be, isolated from a natural source, chemically synthesized or recombinantly produced by any technique known to those of skill in the art. Natural products include such categories as mitotic inhibitors, antitumor antibiotics, enzymes and biological response modifiers.

Mitotic inhibitors include plant alkaloids and other natural agents that can inhibit either protein synthesis required for cell division or mitosis. They operate during a specific phase during the cell cycle. Mitotic inhibitors include, for example, docetaxel, etoposide (VP 16), teniposide, paclitaxel, taxol, vinblastine, vincristine, and vinorelbine.

Taxoids are a class of related compounds isolated from the bark of the ash tree, Taxus brevifolia. Taxoids include but are not limited to compounds such as docetaxel and paclitaxel. Paclitaxel binds to tubulin (at a site distinct from that used by the vinca alkaloids) and promotes the assembly of microtubules.

Vinca alkaloids are a type of plant alkaloid identified to have pharmaceutical activity. They include such compounds as vinblastine (VLB) and vincristine.

7. Antitumor Antibiotics

Antitumor antibiotics have both antimicrobial and cytotoxic activity. These drugs also interfere with DNA by chemically inhibiting enzymes and mitosis or altering cellular membranes. These agents are not phase specific so they work in all phases of the cell cycle. Examples of antitumor antibiotics include, but are not limited to, bleomycin, dactinomycin, daunorubicin, doxorubicin (Adriamycin), plicamycin (mithramycin) and idarubicin.

8. Hormones

Corticosteroid hormones are considered chemotherapy drugs when they are implemented to kill or slow the growth of cancer cells. Corticosteroid hormones can increase the effectiveness of other chemotherapy agents, and consequently, they are frequently used in combination treatments. Prednisone and dexamethasone are examples of corticosteroid hormones.

Progestins such as hydroxyprogesterone caproate, medroxyprogesterone acetate, and megestrol acetate have been used in cancers of the endometrium and breast. Estrogens such as diethylstilbestrol and ethinyl estradiol have been used in cancers such as breast and prostate. Antiestrogens such as tamoxifen have been used in cancers such as breast. Androgens such as testosterone propionate and fluoxymesterone have also been used in treating breast cancer. Antiandrogens such as flutamide have been used in the treatment of prostate cancer. Gonadotropin-releasing hormone analogs such as leuprolide have been used in treating prostate cancer.

9. Miscellaneous Agents

Some chemotherapy agents do not fall into the previous categories based on their activities. They include, but are not limited to, platinum coordination complexes, anthracenedione, substituted urea, methyl hydrazine derivative, adrenalcortical suppressant, amsacrine, L-asparaginase, and tretinoin. It is contemplated that they may be used within the compositions and methods of the present invention.

Platinum coordination complexes include such compounds as carboplatin and cisplatin (cis-DDP).

An anthracenedione such as mitoxantrone has been used for treating acute granulocytic leukemia and breast cancer. A substituted urea such as hydroxyurea has been used in treating chronic granulocytic leukemia, polycythemia vera, essential thrombocytosis and malignant melanoma. A methyl hydrazine derivative such as procarbazine (N-methylhydrazine, MIH) has been used in the treatment of Hodgkin's disease. An adrenocortical suppressant such as mitotane has been used to treat adrenal cortex cancer, while aminoglutethimide has been used to treat Hodgkin's disease.

10. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl.sub.XL, Bcl.sub.W, Bcl.sub.S, Mc1-1, A1, Bfl-1) or counteract Bcl-2 function and promote cell death (e.g., Bax, Bak, Bik, Bim, Bid, Bad, Harakiri).

Non-limiting examples of pro-apoptosis agents contemplated within the scope of the present invention include gramicidin, magainin, mellitin, defensin, and cecropin.

11. Angiogenic Inhibitors

In certain embodiments the present invention may concern administration of targeting peptides attached to anti-angiogenic agents, such as angiotensin, laminin peptides, fibronectin peptides, plasminogen activator inhibitors, tissue metalloproteinase inhibitors, interferons, interleukin 12, platelet factor 4, IP-10, Gro-.beta., thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP470, endostatin, paclitaxel, accutin, angiostatin, cidofovir, vincristine, bleomycin, AGM-1470, platelet factor 4 or minocycline.

12. Dosages

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

B. Imaging Agents

In certain embodiments, the claimed peptides or proteins of the present invention may be attached to imaging agents of use for imaging and diagnosis of various diseased organs, tissues or cell types. Many appropriate imaging agents are known in the art, as are methods for their attachment to proteins or peptides (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the protein or peptide (U.S. Pat. No. 4,472,509). Proteins or peptides also may be reacted with an enzyme in the presence of a coupling agent such as glutaraldehyde or periodate. Conjugates with fluorescein markers are prepared in the presence of these coupling agents or by reaction with an isothiocyanate.

Non-limiting examples of paramagnetic ions of potential use as imaging agents include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly useful. Ions useful in other contexts, such as X-ray imaging, include but are not limited to lanthanum (III), gold (III), lead (II), and especially bismuth (III).

Radioisotopes of potential use as imaging or therapeutic agents include astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and yttrium⁹⁰. ¹²⁵I is often being employed for use in certain embodiments, and technicium^{99m} and indium¹¹¹ are also often utilized due to their low energy and suitability for long range detection.

Radioactively labeled proteins or peptides of the present invention may be produced according to well-known methods in the art. For instance, they can be iodinated by contact with sodium or potassium iodide and a chemical oxidizing agent such as sodium hypochlorite, or an enzymatic oxidizing agent, such as lactoperoxidase. Proteins or peptides according to the invention may be labeled with technetium-^{99m} by ligand exchange process, for example, by reducing pertechnate with stannous solution, chelating the reduced technetium onto a Sephadex column and applying the peptide to this column or by direct labeling techniques, e.g., by incubating pertechnate, a reducing agent such as SNCl₂, a buffer solution such as sodium-potassium phthalate solution, and the peptide. Intermediary functional groups which are often used to bind radioisotopes which exist as metallic ions to peptides are diethylenetriaminepentaacetic acid (DTPA) and ethylene diaminetetracetic acid (EDTA). Also contemplated for use are fluorescent labels, including rhodamine, fluorescein isothiocyanate and renographin.

In certain embodiments, the claimed peptides may be linked to a secondary binding ligand or to an enzyme (an enzyme tag) that will generate a colored product upon contact with a chromogenic substrate. Examples of suitable enzymes include urease, alkaline phosphatase, (horseradish) hydrogen peroxidase and glucose oxidase. Some secondary binding ligands are biotin and avidin or streptavidin compounds. The use of such labels is well known to those of skill in the art in light and is described, for example, in U.S. Pat. Nos. 3,817,837; 3,850,752; 3,939,350; 3,996,345; 4,277,437; 4,275,149 and 4,366,241; each incorporated herein by reference.

### VIII. Other Agents

In some embodiments, the targeting peptide is attached to an agent, such as a macromolecular complex, e.g., virus, a bacteriophage, a bacterium, a liposome, a microparticle, a nanoparticle, a magnetic bead, a yeast cell, a mammalian cell, a cell, an eukaryotic expression vector, quantum dot, phosphorodiamidate morpholino oligomers, or a microdevice. The attachment of the targeting peptide to the macromolecular complex may be a covalent attachment or a non-covalent attachment. Macromolecular complexes within the scope of the present invention include virtually any macromolecular complex that may be associated with (such as attached to) a targeting peptide and administered to a subject.

A. Nanoparticles

In some embodiments, the targeting peptide is attached to a nanoparticle. The term “nanoparticle” denotes a microscopic carrier structure that is able to have a peptide directly or indirectly attached thereto and is biocompatible. In some embodiments, the nanoparticle is sufficiently resistant to chemical and/or physical destruction by the environment of use such that a sufficient amount of the nanoparticles remain substantially intact after delivery into an individual. Nanoparticles can be solid colloidal particles ranging in size from 1 to 1000 nm. Nanoparticle can have any diameter less than or equal to 1000 nm, including less than or equal to 5, 10, 15, 20, 25, 30, 50, 100, 500 and 750 nm. Peptides can be incubated with the nanoparticles and thereby be adsorbed or attached to the nanoparticle, in some embodiments. For example, peptides have been coupled first to bovine serum albumin (BSA) via the bifunctional crosslinker 3-maleimido benzoic acid N-hydroxysuccinimide (MBS) and then attached to gold nanoparticles via electrostatic interactions. In some embodiments, there is covalent coupling of cysteine-terminated peptides directly to a gold particle surface via a sulfur-gold bond. In some embodiments, peptide-coated iron oxide nanoparticles are employed.

In some embodiments, nanoparticles are comprised of, e.g., metal, carbon, graphite or polymer. The nanoparticle can comprise a variety of inorganic materials including, but not limited to, metals, semi-conductor materials or ceramics. Preferred metal-based compounds for the manufacture of nanoparticles include titanium, titanium dioxide, tin, tin oxide, silicon, silicon dioxide, iron, iron.sup.111 oxide, silver, gold, copper, nickel, aluminum, steel, cobalt-chrome alloys, cadmium (such as cadmium selenide) and titanium alloys. Ceramic materials include brushite, tricalcium phosphate, alumina, silica, and zirconia. The nanoparticle can be made from organic materials including carbon (diamond). Exemplary polymers include polystyrene, silicone rubber, polycarbonate, polyurethanes, polypropylenes, polymethylmethacrylate, polyvinyl chloride, polyesters, polyethers, and polyethylene. In some embodiments, gold is useful because of its well-known reactivity profiles and biological inertness.

B. Liposomes

In some embodiments, the peptide is directly or indirectly linked to a liposome. A liposome used for the preparation of a vehicle of the invention is, in simplest form, composed of two lipid layers. The lipid layer may be a monolayer, or may be multilamellar and include multiple layers. Constituents of the liposome may include, for example, phosphatidylcholine, cholesterol, phosphatidylethanolamine, and so forth. Phosphatidic acid, which imparts an electric charge, may also be added. Peptides may be conjugated to liposomes by conjugation, in some embodiments.

The liposome may be comprised of phosphatidylcholine, but can include various natural (e.g., tissue derived L-α-phosphatidyl: egg yolk, heart, brain, liver, soybean) and/or synthetic (e.g., saturated and unsaturated 1,2-diacyl-SN-glycero-3-phosphocholines, 1-acyl-2-acyl-SN-glycero-3-phosphocholines, 1,2-diheptanoyl-SN-glycero-3-phosphocholine) derivatives of the same. Such lipids can be used alone, or in combination with a helper lipid. Some helper lipids are non-ionic or uncharged at physiological pH. Particularly preferred non-ionic lipids include, but are not limited to, cholesterol and DOPE (1,2-dioleolylglyceryl phosphatidylethanolamine), with cholesterol being most preferred. The molar ratio of a phospholipid to helper lipid can range from about 3:1 to about 1:1, more preferably from about 1.5:1 to about 1:1, and most preferably, the molar ratio is about 1:1. Combining the Targeting Agent and Active Agent

In some embodiments, a targeting agent is utilized with an active agent. The two moieties may be employed in any manner so long as they are able to be delivered to the target site such that the active agent can act at or near the target site, including an endothelial target site. The two moieties may be combined covalently or non-covalently. In some embodiments, the two moieties are conjugated. In other aspects, the two moieties are cross-linked.

Bifunctional cross-linking reagents have been extensively used for a variety of purposes including preparation of affinity matrices, modification and stabilization of diverse structures, identification of ligand and receptor binding sites, and structural studies. Homobifunctional reagents that carry two identical functional groups proved to be highly efficient in inducing cross-linking between identical and different macromolecules or subunits of a macromolecule, and linking of polypeptide ligands to their specific binding sites. Heterobifunctional reagents contain two different functional groups. By taking advantage of the differential reactivities of the two different functional groups, cross-linking can be controlled both selectively and sequentially. The bifunctional cross-linking reagents can be divided according to the specificity of their functional groups, e.g., amino, sulfhydryl, guanidino, indole, carboxyl specific groups. Of these, reagents directed to free amino groups have become especially popular because of their commercial availability, ease of synthesis and the mild reaction conditions under which they can be applied. A majority of heterobifunctional cross-linking reagents contains a primary amine-reactive group and a thiol-reactive group.

Exemplary methods for cross-linking ligands to liposomes are described in U.S. Pat. No. 5,603,872 and U.S. Pat. No. 5,401,511, each specifically incorporated herein by reference in its entirety). Various ligands can be covalently bound to liposomal surfaces through the cross-linking of amine residues. Liposomes, in particular, multilamellar vesicles (MLV) or unilamellar vesicles such as microemulsified liposomes (MEL) and large unilamellar liposomes (LUVET), each containing phosphatidylethanolamine (PE), have been prepared by established procedures. The inclusion of PE in the liposome provides an active functional residue, a primary amine, on the liposomal surface for cross-linking purposes. Ligands such as epidermal growth factor (EGF) have been successfully linked with PE-liposomes. Ligands are bound covalently to discrete sites on the liposome surfaces. The number and surface density of these sites are dictated by the liposome formulation and the liposome type. The liposomal surfaces may also have sites for non-covalent association. To form covalent conjugates of ligands and liposomes, cross-linking reagents have been studied for effectiveness and biocompatibility. Cross-linking reagents include glutaraldehyde (GAD), bifunctional oxirane (OXR), ethylene glycol diglycidyl ether (EGDE), and a water soluble carbodiimide, such as 1-ethyl-3-(3-dimethylaminopropyl) carbodiimide (EDC). Through the complex chemistry of cross-linking, linkage of the amine residues of the recognizing substance and liposomes is established.

In another example, heterobifunctional cross-linking reagents and methods of using the cross-linking reagents are described (U.S. Pat. No. 5,889,155, specifically incorporated herein by reference in its entirety). The cross-linking reagents combine a nucleophilic hydrazide residue with an electrophilic maleimide residue, allowing coupling in one example, of aldehydes to free thiols. The cross-linking reagent can be modified to cross-link various functional groups.

### IX. Nucleic Acids

Nucleic acids according to the present invention may encode a targeting peptide, a receptor protein or a fusion protein. The nucleic acid may be derived from genomic DNA, complementary DNA (cDNA) or synthetic DNA. Where incorporation into an expression vector is desired, the nucleic acid may also comprise a natural intron or an intron derived from another gene. Such engineered molecules are sometime referred to as “mini-genes.”

A “nucleic acid” as used herein includes single-stranded and double-stranded molecules, as well as DNA, RNA, chemically modified nucleic acids and nucleic acid analogs. It is contemplated that a nucleic acid within the scope of the present invention may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or greater nucleotide residues in length.

It is contemplated that targeting peptides, fusion proteins and receptors may be encoded by any nucleic acid sequence that encodes the appropriate amino acid sequence. The design and production of nucleic acids encoding a desired amino acid sequence is well known to those of skill in the art, using standardized codon tables. In some embodiments, the codons selected for encoding each amino acid may be modified to optimize expression of the nucleic acid in the host cell of interest. Codon preferences for various species of host cell are well known in the art.

In addition to nucleic acids encoding the desired targeting peptide, fusion protein or receptor amino acid sequence, the present invention encompasses complementary nucleic acids that hybridize under high stringency conditions with such coding nucleic acid sequences. High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleotide content of the target sequence(s), the charge composition of the nucleic acid(s), and to the presence or concentration of formamide, tetramethylammonium chloride or other solvent(s) in a hybridization mixture.

### X. Vectors for Cloning, Gene Transfer and Expression

In certain embodiments expression vectors are employed to express the targeting peptide or fusion protein, which can then be purified and used. In some embodiments, the expression vectors are used in gene therapy as therapeutic agents employed with a targeting agent of the invention. In any event, expression requires that appropriate signals be provided in the vectors, and which include various regulatory elements, such as enhancers/promoters from viral and/or mammalian sources that drive expression of the genes of interest in host cells, such as endothelial cells, for example. Elements designed to optimize messenger RNA stability and translatability in host cells also are known.

A. Regulatory Elements

The terms “expression construct” or “expression vector” are meant to include any type of genetic construct containing a nucleic acid coding for a gene product in which part or all of the nucleic acid coding sequence is capable of being transcribed. In some embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The particular promoter employed to control the expression of a nucleic acid sequence of interest is not believed to be important, so long as it is capable of directing the expression of the nucleic acid in the targeted cell, such as an endothelial cell. Thus, where a human cell is targeted, it is useful to position the nucleic acid coding region adjacent and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

B. Selectable Markers

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. In some embodiments, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

C. Expression Vector Embodiments

There are a number of ways in which expression vectors may introduced into cells, including endothelial cells. In certain embodiments of the invention, the expression construct comprises a virus or engineered construct derived from a viral genome. The ability of certain viruses to enter cells via receptor-mediated endocytosis, to integrate into host cell genome, and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Some gene therapy vectors are generally viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression making them ideally suited for rapid, efficient, heterologous gene expression. Techniques for preeparing replication infective viruses are well known in the art.

In using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A means of purifying the vector involves the use of buoyant density gradients, such as cesium chloride gradient centrifugation.

DNA viruses used as gene vectors include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

In some embodiments, methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include, but is not limited to, constructs containing adenovirus sequences sufficient to (a) support packaging of the construct and (b) to express an antisense or a sense polynucleotide that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retroviral infection, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them useful mRNAs for translation.

In currently used systems, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of adenovirus vectors which are replication deficient depend on a unique helper cell line, designated 293, which is transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3, or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. In some embodiments, the helper cells may be derived from the cells of other mammalian species that are permissive for human adenovirus. Such cells include, e.g., Vero cells or other monkey embryonic mesenchymal or epithelial cells. As discussed, a usefulhelper cell line is 293.

Racher et al., (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) are employed as follows. A cell innoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking is initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking is commenced for another 72 hr.

Other than the embodiment wherein the adenovirus vector is replication defective, or at least conditionally defective, the nature of the adenovirus vector is not believed to be crucial to the successful practice of the invention. The adenovirus may be of any of the 42 different known serotypes or subgroups A-F. In some embodiments, adenovirus type 5 of subgroup C is the starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

A typical vector applicable to practicing the present invention is replication defective and will not have an adenovirus E1 region. Thus, it are most convenient to introduce the polynucleotide encoding the gene at the position from which the E1-coding sequences have been removed. However, the position of insertion of the construct within the adenovirus sequences is not critical. The polynucleotide encoding the gene of interest may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic innoculation into the brain (Le Gal La Salle et al., 1993).

Other gene transfer vectors may be constructed from retroviruses. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env. that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990).

In order to construct a retroviral vector, a nucleic acid encoding protein of interest is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

There are certain limitations to the use of retrovirus vectors. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This may result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), and herpes viruses may be employed. They offer several attractive features for various mammalian cells (Friedmann, 1989; Ridgeway, 1988; Baichwal and Sugden, 1986; Coupar et al., 1988; Horwich et al., 1990).

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990), DEAE-dextran (Gopal, 1985), electroporation (Tur-Kaspa et al., 1986; Potter et al., 1984), direct microinjection, DNA-loaded liposomes and lipofectamine-DNA complexes, cell sonication, gene bombardment using high velocity microprojectiles, and receptor-mediated transfection (Wu and Wu, 1987; Wu and Wu, 1988). Some of these techniques may be successfully adapted for in vivo or ex vivo use.

In some embodiments, the expression construct is entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection.

A number of selection systems may be used including, but not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr: that confers resistance to methotrexate; gpt, that confers resistance to mycophenolic acid; neo, that confers resistance to the aminoglycoside G418; and hygro, that confers resistance to hygromycin.

### XI. Kits of the Invention

Any of the compositions described herein may be comprised in a kit. In a non-limiting example, a targeting agent and/or active agent, including a therapeutic or diagnostic agent, such as an imaging agent, may be comprised in a kit in suitable container means.

The kits may comprise a suitably aliquoted targeting agent and/or active agent of the present invention. The components of the kits may be packaged either in aqueous media or in lyophilized form. However, the components of the kit may be provided as dried powder(s). When reagents and/or components are provided as a dry powder, the powder can be reconstituted by the addition of a suitable solvent. It is envisioned that the solvent may also be provided in another container means.

The container means of the kits will generally include at least one vial, test tube, flask, bottle, syringe or other container means, into which a component may be placed, and in some embodiments, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the targeting peptide and/or active agent and any other reagent containers in close confinement for commercial sale. Such containers may include injection or blow molded plastic containers into which the desired vials are retained. The container means may itself be a syringe, pipette, and/or other such like apparatus, from which the formulation may be applied to an affected area of the body, injected into an animal, and/or even applied to and/or mixed with the other components of the kit.

### EXAMPLES

The following examples are included to demonstrate some embodiments of the invention. It should be appreciated by those of skill in the art that the techniques disclosed in the examples which follow represent techniques discovered by the inventor to function well in the practice of the invention, and thus can be considered to constitute some modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification of Targeting Agents

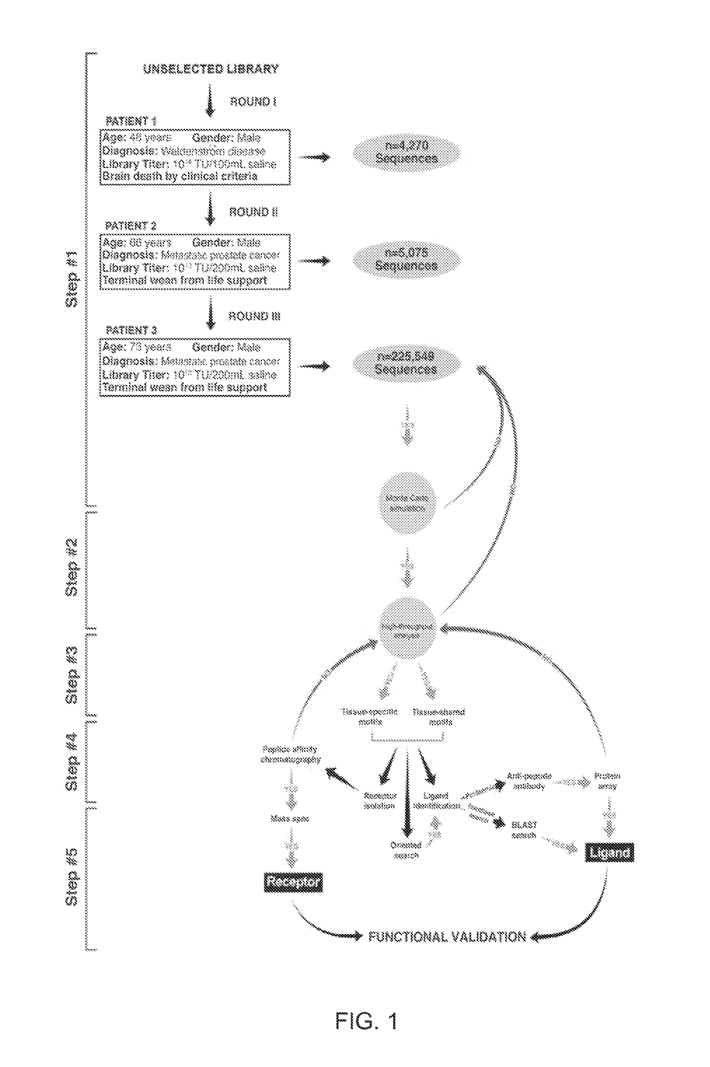

The inventors applied serial rounds of direct combinatorial selection in three cancer patients. As an outline (FIG. 1), after systemic delivery of a phage-displayed random CX7C (C, cysteine; X, any residue) cyclic peptide library to the first human subject (Arap et al., 2002), ligand phage populations were recovered, pooled, and serially screened in two subsequent patients (FIG. 1, Step #1). To evaluate putative interactions identified with this approach, virtual filters were designed to streamline the large-scale process of candidate ligand selection of biologically active peptides, and there was subsequent discovery and validation with native circulating ligands and corresponding receptors. The inventors developed a comprehensive bioinformatics approach to systematically assess targeted phage particle distribution and potential tissue-specificity by means of a custom-designed algorithm (Kolonin et al., 2006a) to perform Monte Carlo simulation on large peptide datasets (FIG. 1, Step #2). Predicted selection of targeting peptides over the initial (random) library was followed by a high-throughput analysis of tripeptide motifs. With the incorporation of a software program developed in-house (Arap et al., 2002; Kolonin et al., 2006a; Kolonin et al., 2006b) an exhaustive residue count was executed, which kept track of relative frequencies of n distinct motifs representing all possible n3 (any possible combination of three amino acids) overlapping sequences in both directions (n<<n3). The significance of association of a given allocation of counts was assessed by Fisher's exact test (one-tailed), and full 7-mer peptides containing specific tripeptides were identified in the original dataset. Positive selection of targeting motifs could lead to identification of both tissue-specific and shared vascular motifs (FIG. 1, Step #3). In contrast, negative selection at this stage of evaluation would suggest that the raw data be revisited for quality assurance and quality control. Of note, both datasets—Monte Carlo simulations and high-throughput analyses—indicated a favorable chance of selection and the identification of targeting peptides.

Three non-mutually exclusive approaches were subsequently applied to the isolation of functional ligand-receptor pairs (FIG. 1, Step #4): (i) protein purification and identification of candidate receptors for each motif of interest were performed by in tandem affinity chromatography and mass spectrometry, (ii) identification of putative native ligands for the isolated receptors was achieved from protein array screenings with anti-peptide antibodies or basic local alignment and search tool (BLAST) analysis, and (iii) supervised online protein database searches served to evaluate known ligand-receptor interactions. Ultimately, functional validation of ligand-receptor systems through competitive binding assays was performed in the appropriate biochemistry settings (FIG. 1, Step #5).

Three rounds of synchronous combinatorial screening in patients produced a total of 2,348,940 tripeptide motif sequences (each 7-mer phage-displayed insert contributed five potential overlapping tripeptides in each direction), allowing assessment of virtually every peptide displayed in the selected tissues. For each round, the inventors analyzed frequencies and tissue distribution of all tripeptide motifs in each direction within the recovered full peptide sequences. The same procedure was applied to the parental (unselected) phage display random peptide library. Peptide sequences were evaluated by one-sided Fisher's exact tests, which identified a pool of tripeptide motifs (n=23) significantly enriched (P<0.05) in targeted tissues in round three relative to their original frequencies in the random library prior to selection.

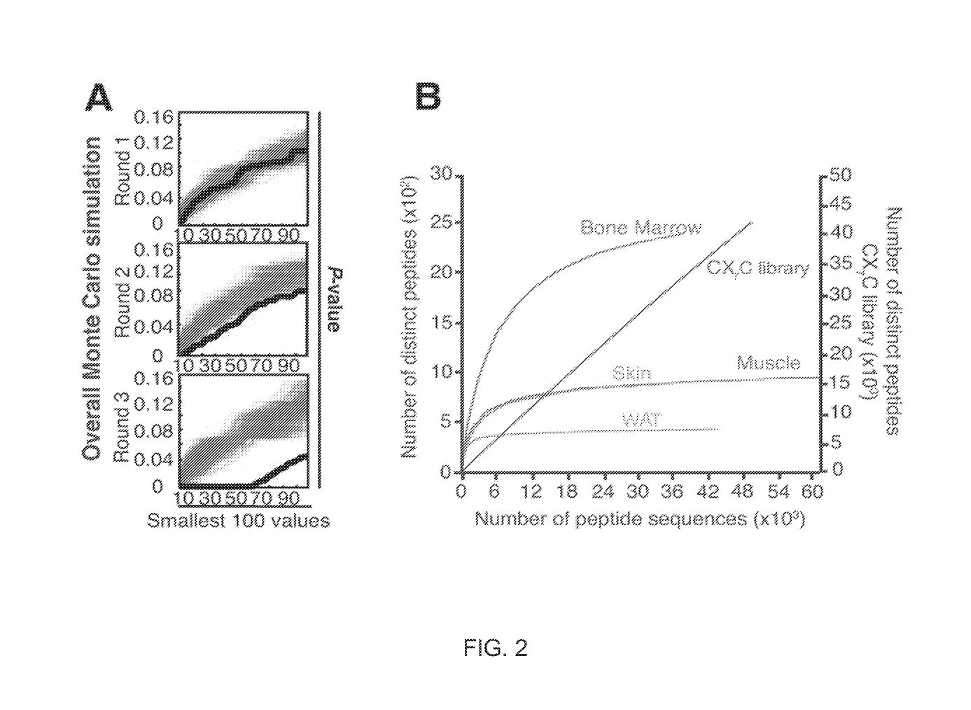

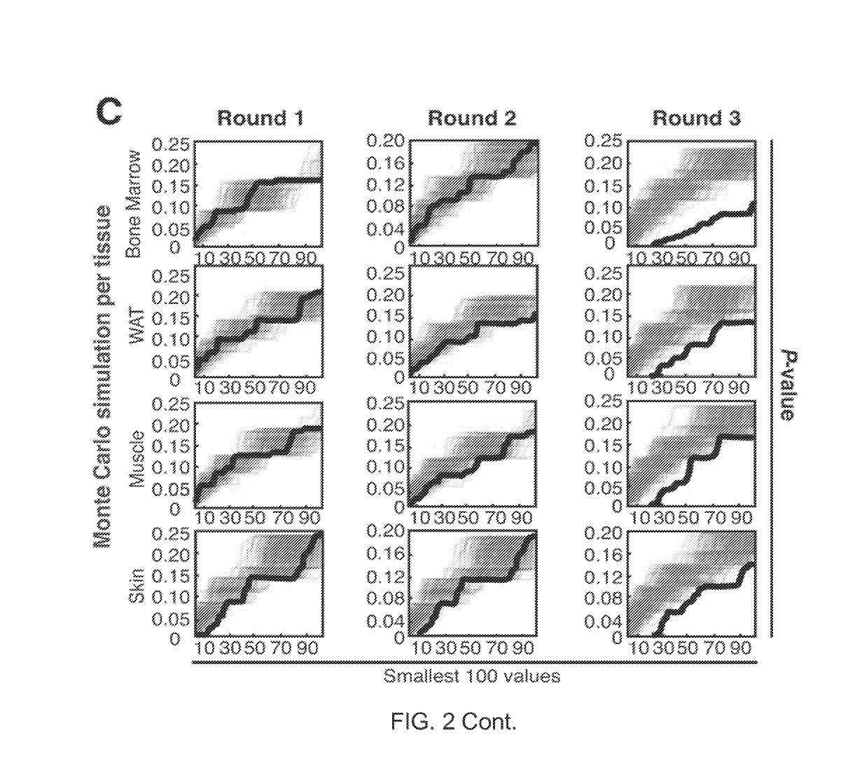

Analysis of large-scale derived sequences established a non-random distribution of tripeptide motifs among tissues (FIG. 2A) with specific saturation curves of ligand peptides (FIG. 2B), and revealed ˜78% agreement between the two different DNA sequencing methods used in this study. In a recent rigorous comparison the inventors determined the superiority of real-time quantitative PCR (termed qPhage) plus DNA pyrosequencing, compared to conventional transducing unit (TU)-counting plus Sanger DNA sequencing, in terms of accuracy and time- or cost-effectiveness for phage display-based applications (see Dias-Neto et al., 2009 and PCT Application Serial No. PCT/US10/61129, incorporated by reference herein in its entirety).

Monte Carlo simulations confirmed a progressive accumulation of enriched motifs from the first to the third round (FIG. 2A), in a tissue-specific manner (FIG. 2C); such simulations also revealed that the analytical design used had a >95% probability of detecting significantly enriched motifs (P<0.05). Saturation plots of high-throughput DNA pyrosequencing performed in the third round confirmed the likely identification of most phage-displayed inserts present within the selected tissues (FIG. 2B). Moreover, each tissue had a specific and reproducible ligand saturation curve, a result suggestive of intrinsic tissue diversity among vascular receptor pools. In contrast, ligand saturation could not be reached with the unselected library under the same experimental conditions, an indication of true randomness (FIG. 2B).

Next, by comparing tripeptide motif frequencies in targeted versus non-targeted tissues, these motifs were not evenly distributed and some were actually enriched with tissue-specificity. Of note, 9 of 23 motifs (39%) recovered from white adipose tissue (WAT) were found only in WAT, 14 of 17 bone marrow-homing motifs (82%) were found only in bone marrow, and all of the smaller number of tripeptide motifs selected in skin and muscle were unique to the tissue-of-origin, a confirmation of enrichment across the selection. Full peptide sequences with apparent tissue-specificity (for tumor-containing bone marrow, skin, WAT, and muscle) were obtained from selected tripeptide motifs. Standard biochemistry and/or similarity searches with peptide-based affinity chromatography and mass spectrometry methodologies were systematically used to uncover candidate native ligand-receptors for each selected peptide in the human vasculature. Additionally, protein array screenings with polyclonal antibodies raised against the targeting peptides and similarity data mining have also guided the identification of putative native ligands. In aspects of the invention, some of the ligand peptides mimic circulating proteins that bind to receptors exposed on vascular endothelial cells. Thus, the similarity searches were further refined according to protein structure, glycosylation state, or other post-translational modifications, as well as subcellular location and membrane orientation. As a functional proof-of-concept (i.e., specific binding), there are four extended examples of ligand-receptor systems in shared (n=2) and tissue-specific (n=2) settings selected from human blood vessels of both normal (n=3) and tumor-containing (n=1) tissues. The successful validation of these four candidate ligand-receptor pairs represents only a small fraction of the translation potential of this technology.

### Example 2

### Selection and Validation of a Ligand-Receptor System in Human Wat

In previous research with a combinatorial library, the inventors had found that the peptide CKGGRAKDC (SEQ ID NO:1) was localized to and internalized by cells of mouse WAT vasculature, and that its native vascular target was prohibitin, a protein expressed selectively on mouse and human WAT endothelium (Kolonin et al., 2004).

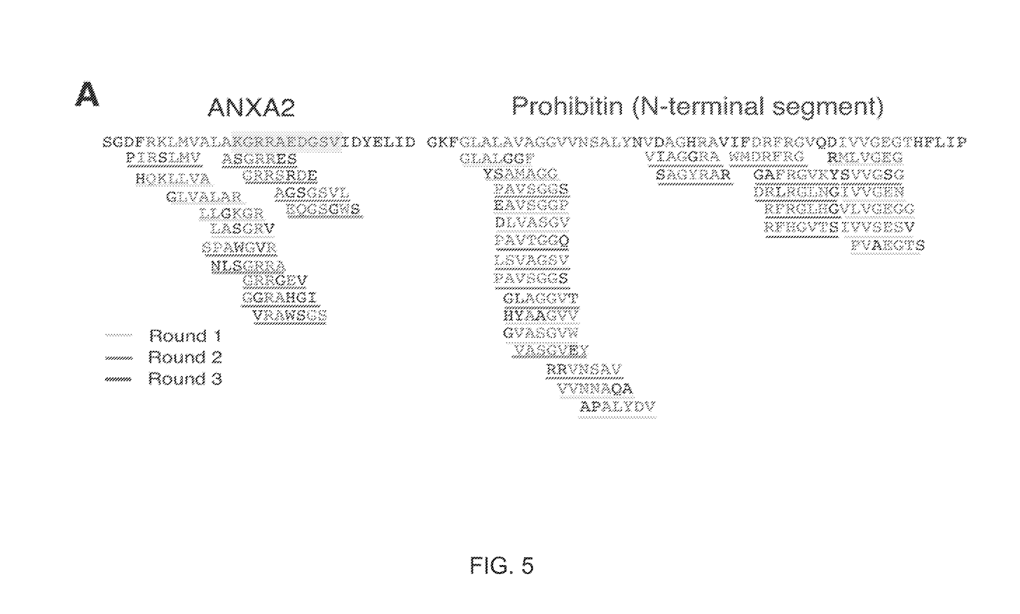

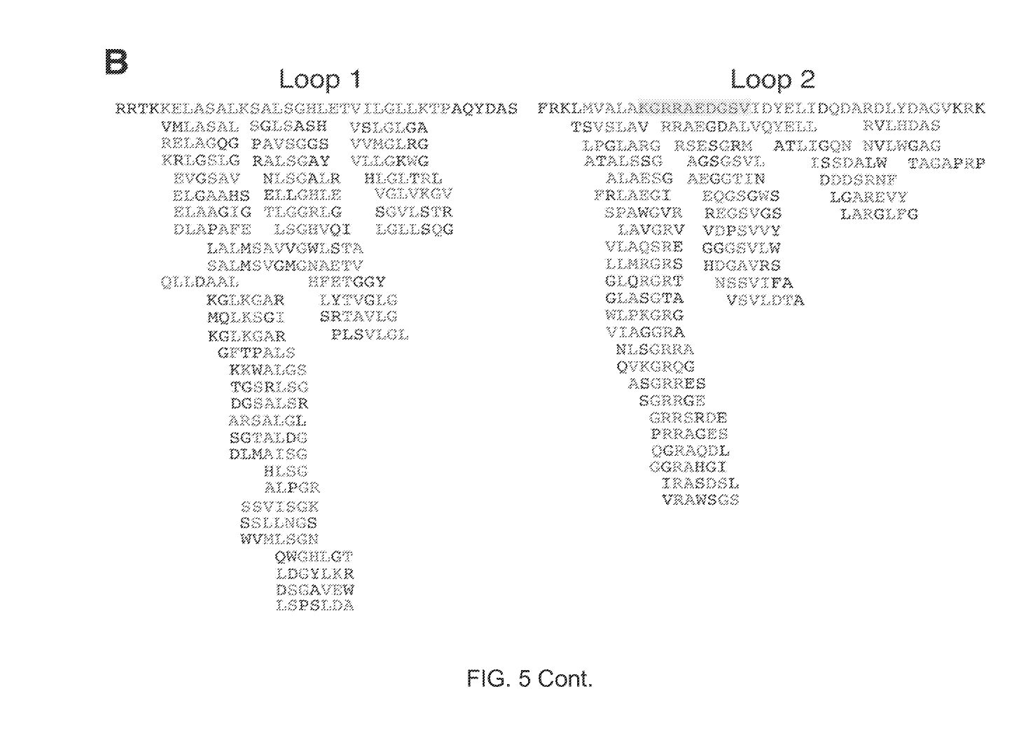

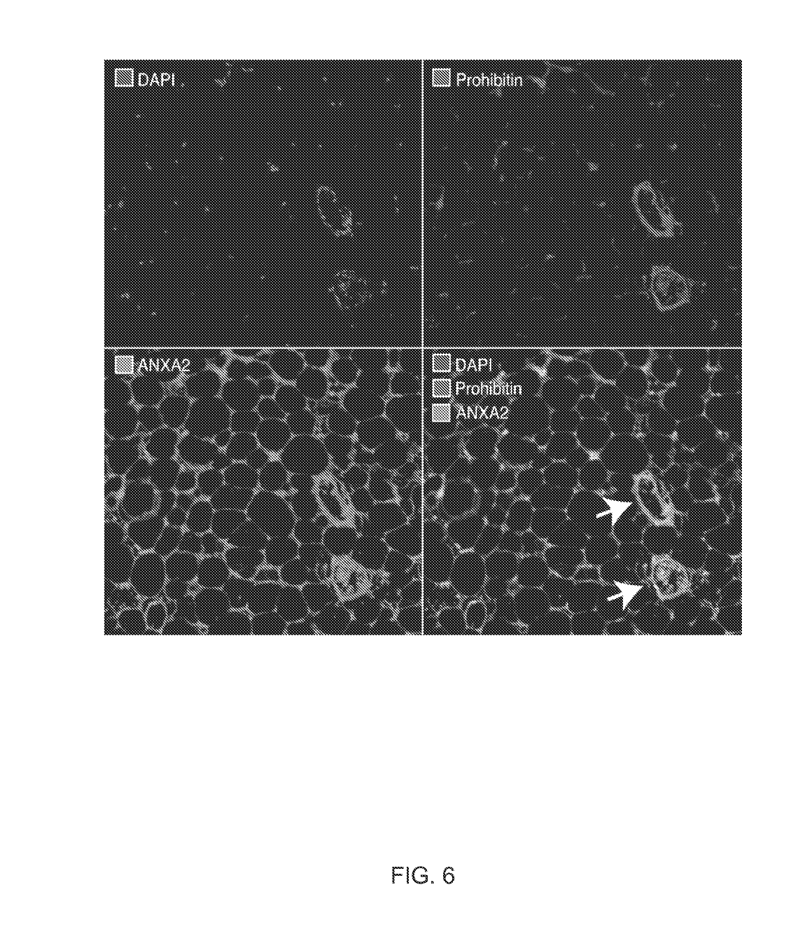

Given that mouse and human prohibitin differ by only a single residue, it was considered whether this protein also serves as a target for WAT-homing peptides in patients, and evaluated this possibility in the present study by analysis of the binding of a large pool of peptide-targeted phage clones (n=851) isolated after three rounds of selection in human WAT to bind to immobilized prohibitin. A subset of non-redundant peptide sequences (n=66) that showed specific binding to prohibitin was subjected to individual database searches for similarity to human proteins, leading to identification of a smaller subset of peptides (n=14) similar to the prohibitin-binding sequence CKGGRAKDC (SEQ ID NO:1) that matched a region of human annexin A2 (ANXA2) (FIG. 5A). Reciprocal analysis revealed a wide range of sequences among all human WAT-selected peptides that mimicked prohibitin and that were clustered within the prohibitin N-terminal segment (FIG. 5A; right), a region involved in lipid raft location and interaction with other proteins, including ANXA2 (Liu et al., 2005). Exemplary ANXA2-mimicking peptide sequences listed therein that may be employed in the invention include at least the following: ASGRRES (SEQ ID NO:14); HOKLLVA (SEQ ID NO:15); GRRSRDE (SEQ ID NO:16); GLVALAR (SEQ ID NO:17); AGSGSVL (SEQ ID NO:18); LLGKGR (SEQ ID NO:19); LASGRV (SEQ ID NO:20); SPAWGVR (SEQ ID NO:21); NLSGRRA (SEQ ID NO:22); GRRGEV (SEQ ID NO:23); and GGRAHGI (SEQ ID NO:24). Exemplary prohibitin (N-terminal segment)-mimicking peptide sequences that may be employed in the invention include at least the following: GLALGGF (SEQ ID NO:25); YSAMAGG (SEQ ID NO:26); EAVSGGP (SEQ ID NO:27); DLVASGV (SEQ ID NO:28); PAVTGGQ (SEQ ID NO:29); GLAGGVT (SEQ ID NO:30); HYAAGVV (SEQ ID NO:31); GVASGVW (SEQ ID NO:32); RRVNSAV (SEQ ID NO:33); VVNNAQA (SEQ ID NO:34); APALYDV (SEQ ID NO:35); VIAGGRA (SEQ ID NO:36); GAFRGVK (SEQ ID NO:37); RFHGVTS (SEQ ID NO:38); RMLVGEG (SEQ ID NO:39); GIVVGEN (SEQ ID NO:40); VLVGEGG (SEQ ID NO:41); IVVSESV (SEQ ID NO:42); and PVAEGTS (SEQ ID NO:43).

To map the ANXA2 domains involved in the binding to prohibitin, the inventors next performed automated alignment of human WAT-homing peptides against ANXA2 and identified two similarity hotspots in the N-terminal domain of the protein (FIG. 5B). Strikingly, these segments correspond to the connector loops between ANXA2 repeats 1/2 and 2/3, which are known to interact with membrane-bound proteins (Liu et al., 2005). Exemplary ANXA2 surface exposed connector loop-mimicking peptides enriched in white adipose tissue are as follows for Loop 1: VMLASAL (SEQ ID NO:44); RELAGQG (SEQ ID NO:45); KRLGSLG (SEQ ID NO:46); EVGSAV (SEQ ID NO:47); ELAAGIG (SEQ ID NO:48); DLAPAFE (SEQ ID NO:49); SGLSASH (SEQ ID NO:50); RALSGAY (SEQ ID NO:51); ELLGHLE (SEQ ID NO:52); TLGGRLG (SEQ ID NO:53); LSGHVQI (SEQ ID NO:54); VSLGLGA (SEQ ID NO:55); VVMGLRG (SEQ ID NO:56); VLLGKWG (SEQ ID NO:57); HLGLTRL (SEQ ID NO:58); VGLVKGV (SEQ ID NO:59); SALMSVG (SEQ ID NO:60); QLLDAAL (SEQ ID NO:61); HFETGGY (SEQ ID NO:62); KGLKGAR (SEQ ID NO:63); LYTVGLG (SEQ ID NO:64); MQLKSGI (SEQ ID NO:65); SRTAVLG (SEQ ID NO:66); KGLKGAR (SEQ ID NO:67); TGSRLSG (SEQ ID NO:68); SGTALDG (SEQ ID NO:69); DLMAISG (SEQ ID NO:70); ALPGR (SEQ ID NO:71); SSVISGK (SEQ ID NO:72); SSLLNGS (SEQ ID NO:73); WVMLSGN (SEQ ID NO:74); and LDGYLKR (SEQ ID NO:75). Mimicking peptides for Loop 2 are as follows: TSVSLAV (SEQ ID NO:76); LPGLARG (SEQ ID NO:77); ALAESG (SEQ ID NO:78); SPAWGVR (SEQ ID NO:79); LAVGRV (SEQ ID NO:80); LLMRGRS (SEQ ID NO:81); GLQRGRT (SEQ ID NO:82); GLASGTA (SEQ ID NO:83); WLPKGRG (SEQ ID NO:84); VIAGGRA (SEQ ID NO:85); NLSGRRA (SEQ ID NO:86) QVKGRQG (SEQ ID NO:87); ASGRRES (SEQ ID NO:88); SGRRGE (SEQ ID NO:89); GRRSRDE (SEQ ID NO:90); PRRAGES (SEQ ID NO:91); QGRAQDL (SEQ ID NO:92); GGRAHGI (SEQ ID NO:93); IRASDSL (SEQ ID NO:94); RRAEGDA (SEQ ID NO:95); LVQYELL (SEQ ID NO:96); AGSGSVL (SEQ ID NO:97); AEGGTIN (SEQ ID NO:98); REGSVGS (SEQ ID NO:99); HDGAVRS (SEQ ID NO:100); VSVLDTA (SEQ ID NO:101); ATLIGQN (SEQ ID NO:102); LARGLFG (SEQ ID NO:103); and TAGAPRP (SEQ ID NO:104).

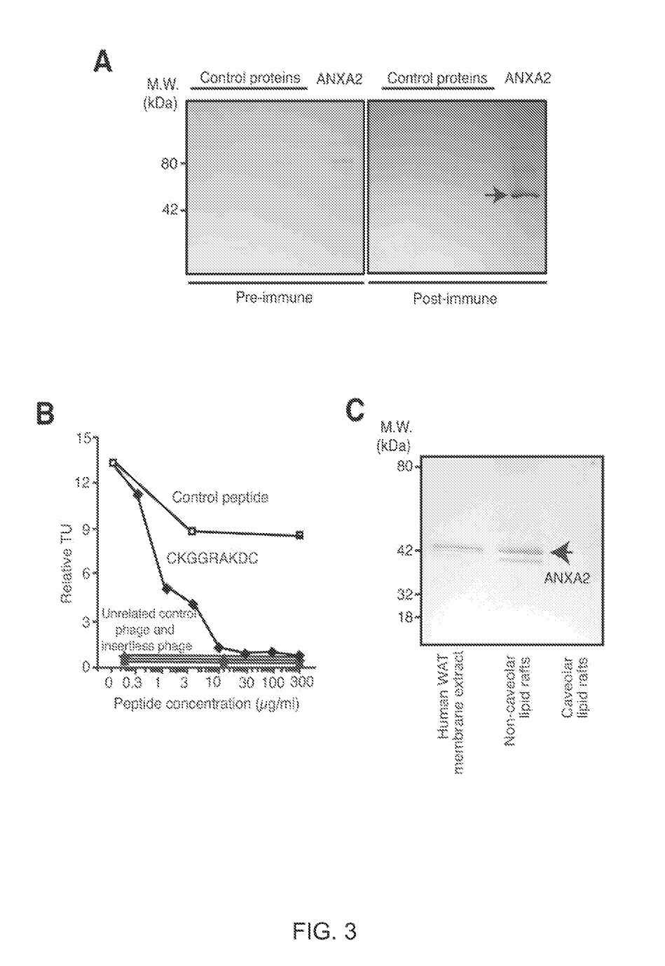

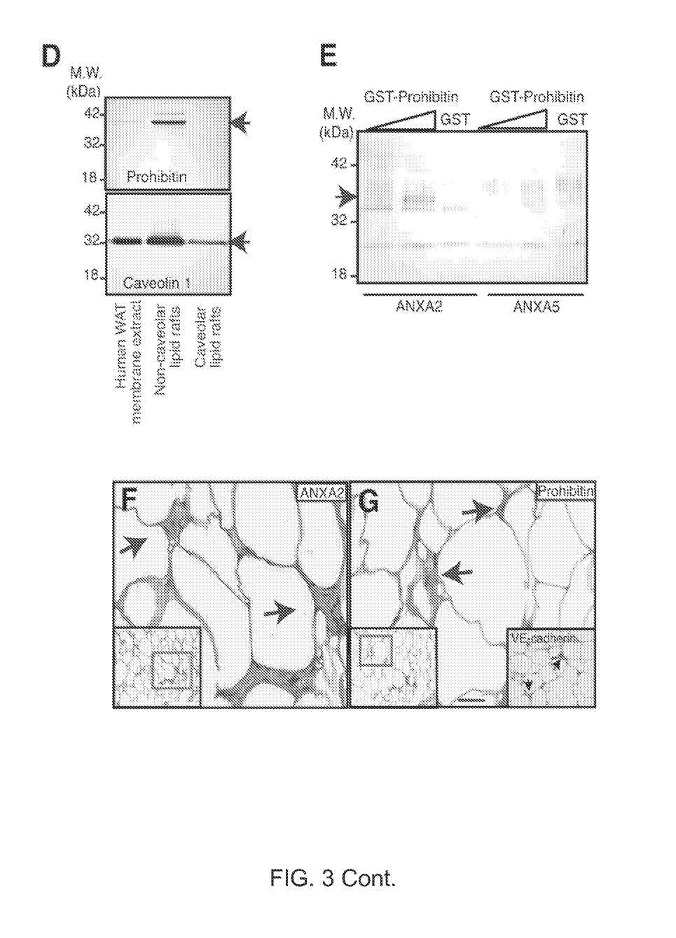

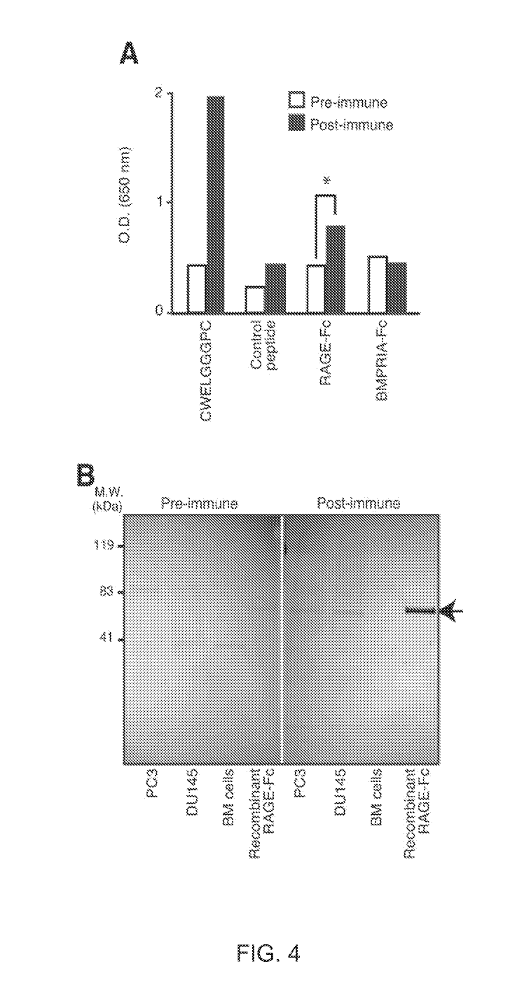

To confirm that CKGGRAKDC (SEQ ID NO:1) mimics the candidate native ligand ANXA2 and binds to prohibitin, antibodies were produced against KLH-conjugated CKGGRAKDC (SEQ ID NO:1), and they recognized recombinant ANXA2 (FIG. 3A). CKGGRAKDC-displaying phage also bound to prohibitin in vitro, an interaction specifically inhibited in a concentration-dependent manner by the cognate synthetic peptide (FIG. 3B). Next, membrane fractions extracted from WAT (FIG. 3C) demonstrated that ANXA2 and prohibitin are located in non-caveolar lipid rafts (FIG. 3D). Lastly, the inventors used recombinant glutathione S-transferase (GST)-conjugated protein to show that prohibitin binds to ANXA2, but not to the control protein ANXAS (FIG. 3E).

Expression of ANXA2 on the surface of endothelial cells has been reported (Ling et al., 2004; Zhang and McCrae, 2005), but without organ comparisons. The expression pattern of prohibitin and ANXA2 in human tissue samples appeared coincident by immunostaining in human WAT (FIGS. 3F and G, and FIG. 6) but not in non-WAT human organs. Taken together, the vascular co-expression and the native ligand-receptor interaction between ANXA2 and prohibitin are restricted to WAT, in at least certain embodiments.

### Example 3

### Discovery of a Specific Ligand-Receptor Pair in Tumor-Containing Human Bone Marrow

Human bone marrow is often affected by primary hematologic tumors (such as leukemias, lymphomas, and myelomas) or metastatic solid tumors (such as breast and prostate carcinomas). In all three patients selected in this study, the bone marrow was replete with tumor cells. Within the bone marrow microenvironment, the inventors assumed that the molecular crosstalk between non-malignant cells of the vascular endothelium and cancer cells might have at least some common elements, independent of the tumor type.

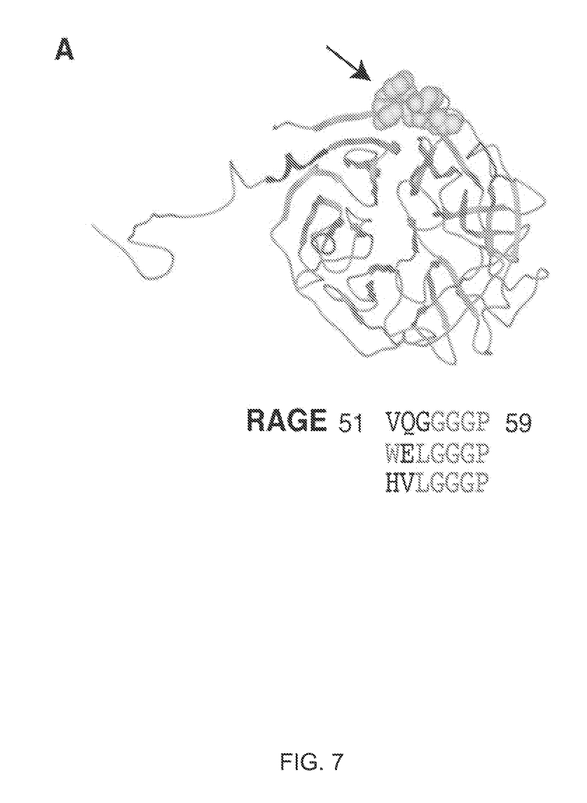

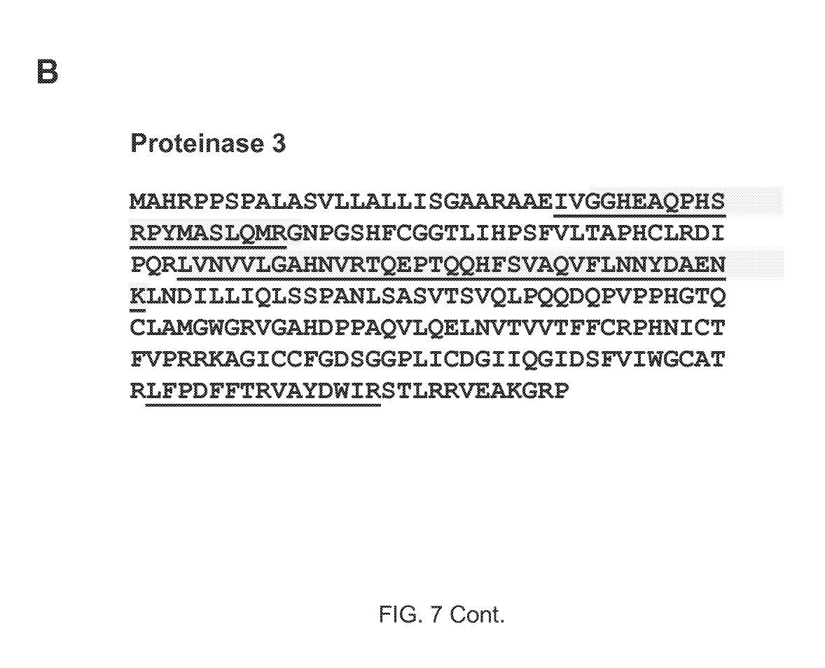

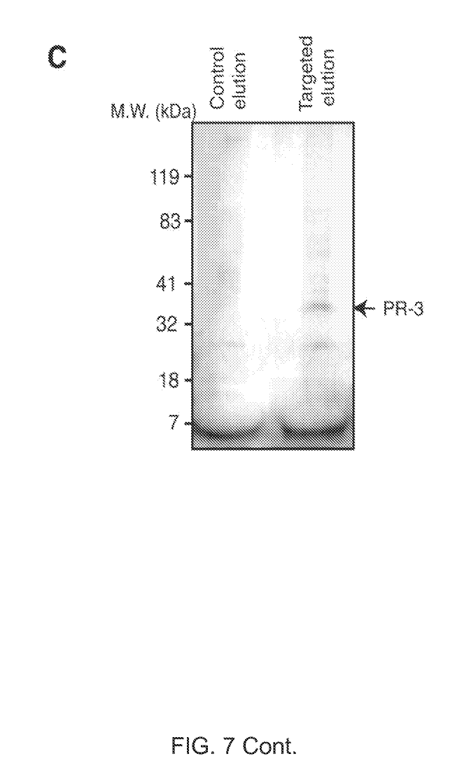

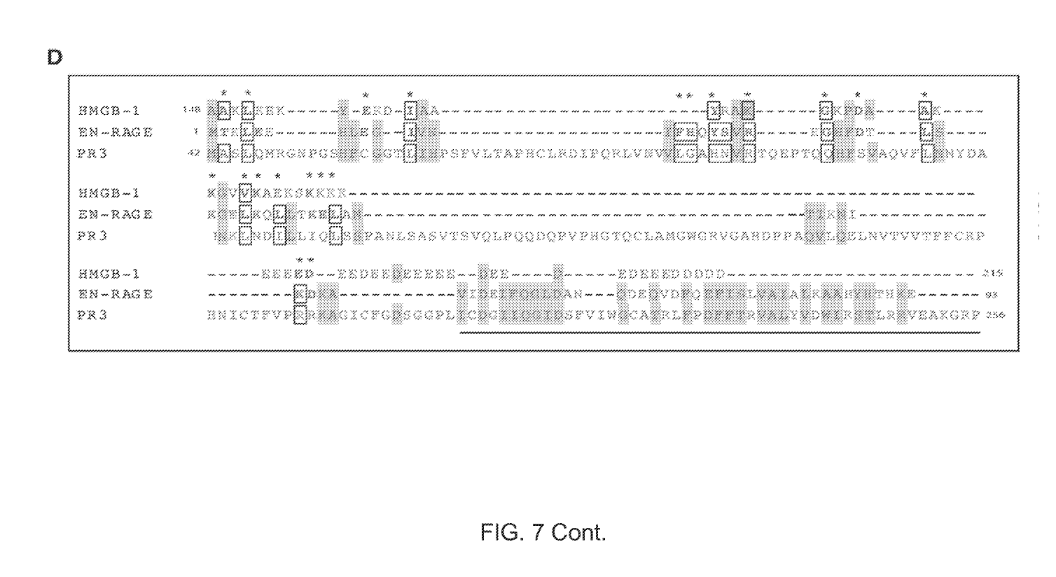

Statistical analysis revealed tripeptides enriched in bone marrow after three rounds of selection, a result consistent with a conserved ligand-receptor system. In particular, selected peptides containing the motif Gly-Gly-Gly-Pro were identified within RAGE, the receptor for advanced glycation end-products. A computer-assisted molecular modeling of RAGE and three 7-mer peptides containing an embedded Gly-Gly-Gly-Pro motif (CWELGGGPC (SEQ ID NO:2), CHVLGGGPC (SEQ ID NO:3), and CVQGGGGPC(SEQ ID NO:4)) showed high similarity to an exposed surface of the ligand-binding extracellular domain of the protein (FIG. 7A).

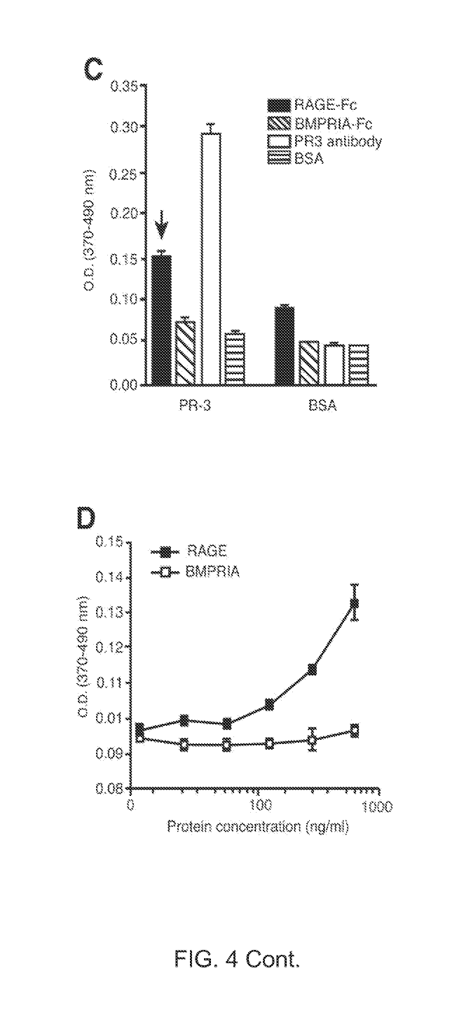

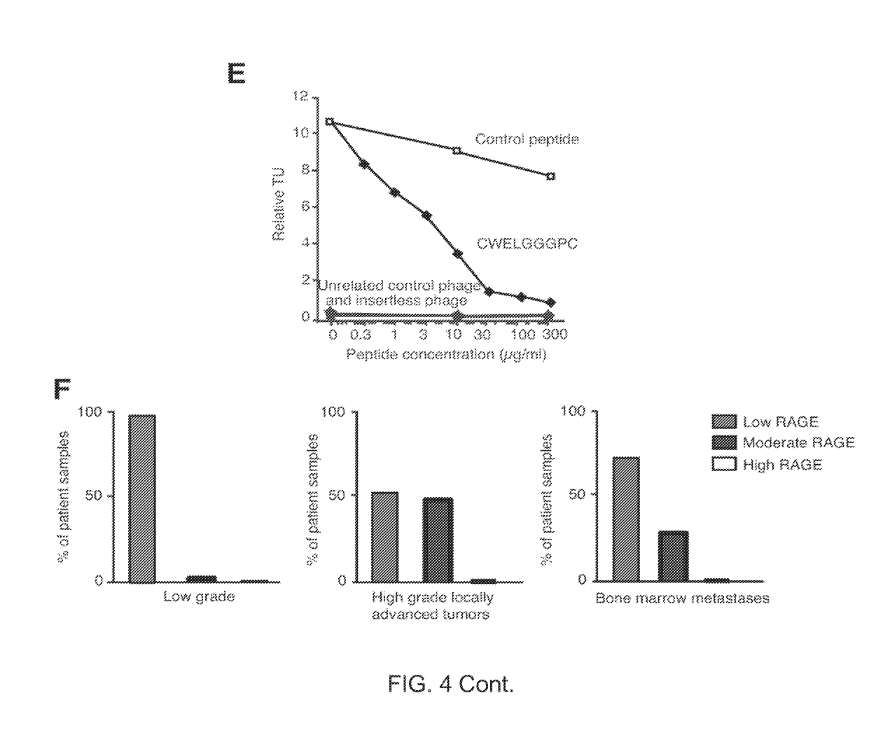

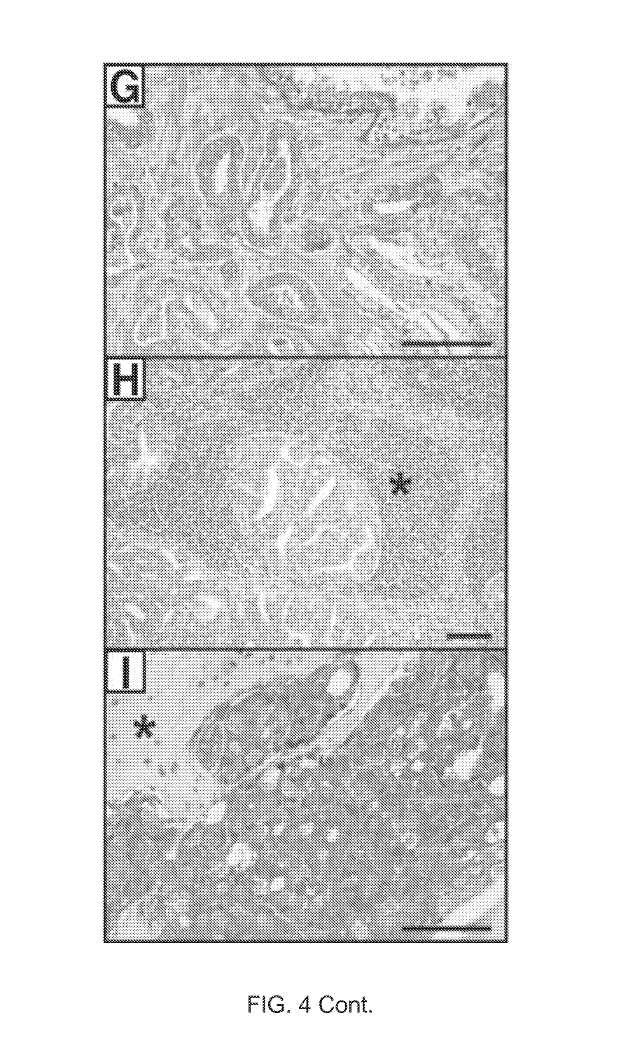

To query whether CWELGGGPC (SEQ ID NO:2) behaves as a molecular mimic of RAGE, a polyclonal antibody was developed against KLH-conjugated CWELGGGPC and used in ELISA to evaluate binding to immobilized RAGE. The anti-CWELGGGPC antibody recognizes the segment of RAGE containing CWKLGGGPC (SEQ ID NO:5; FIG. 4A), whereas pre-immune serum produces only a background signal. Immunoblotting with the anti-CWKLGGGPC antibody confirmed reactivity with the native protein extracted from human prostate cancer cells (FIG. 4B, arrow).

Next, the human leukocyte proteinase-3 (PR-3) was identified by peptide column affinity chromatography and mass spectrometry as a candidate receptor for the targeting peptides (FIG. 7B). This result was confirmed through a second affinity purification with human bone marrow cell membrane extracts as the protein source (FIG. 7C). While PR-3 is a serine protease abundant within the bone marrow microenvironment in patients with chronic myelogenous leukemia (Molldrem et al., 2000), it has not been previously implicated in metastases to bone. In further support of the working hypothesis that PR-3 interacts with RAGE, protein sequence analysis demonstrated that PR-3 does share epitopes with other established RAGE ligands (Campanelli et al., 1990; Sturrock et al., 1992), such as the advanced glycosylation end-products (AGE), high mobility group protein B1 (HMGB1), and 5100 calcium-binding protein A12 (EN-RAGE) (FIG. 7D). Indeed, 7 of 15 HMGB1 residues (47%) and 13 of 21 EN-RAGE residues (60%) critical for RAGE binding (Huttunen et al., 2002) were either identical or conserved within PR-3. Moreover, the C-terminal α-helix of PR-3 is highly similar to the corresponding part of EN-RAGE (FIG. 7C), revealing a previously unrecognized structural relationship between human PR-3 and RAGE partners.

For functional characterization of this putative ligand-receptor pair, the inventors performed binding experiments in vitro between the extracellular portion of human RAGE and endogenous PR-3. RAGE—but not control proteins—bound to immobilized PR-3.

Binding between PR-3 and RAGE is concentration-dependent (FIG. 4D) and competition assays with targeted phage and the cognate synthetic peptide (FIG. 4E) support the specificity of this molecular interaction. These results indicate that the selected human bone marrow-targeting motif mimics a functional site within the extracellular domain of RAGE and that RAGE binds to PR-3 through its WKLGGGP-spanning region. Interestingly, elevation of RAGE mRNA transcripts has been reported in human prostate cancer (Ishiguro et al., 2005), and the expression of RAGE in prostate cancer patients has been documented at the protein level (see the Protein Atlas website). To evaluate this possibility further, the inventors assessed RAGE expression in a large and clinically well-annotated panel of human tumor samples (n=164) from prostate cancer patients (FIG. 4F), including low-grade (n=76) and high-grade locally advanced primary tumors (n=76), and prostate cancer-infiltrated bone marrow biopsy samples (n=12). A linear regression model was applied to assess biomarker expression and distribution among the groups. Significant differences in RAGE expression were observed between low-grade (FIG. 4F, left panel) and high-grade (FIG. 4F, middle panel) tumors (t-test, P<0.0001). Moreover, expression of RAGE was significantly higher in bone marrow-infiltrated metastases compared to low-grade tumors (FIG. 4F, right and left panels; t-test, P=0.0002 for the black bars). There were not statistically significant differences between high-grade primary tumors and bone marrow metastases (t-test, P=0.61). Finally, the inventors detailed RAGE expression immunohistochemically in human prostate cancer, in a representative patient sample set (n=12) including primary tumors (FIG. 4G), lymph node metastases (FIG. 4H), and bone marrow metastases (FIG. 31). While RAGE expression was barely detectable in the normal prostate glands evaluated (FIG. 4G), it was strongly expressed and widespread in tumor cells within the marrow cavity of all cases of prostate cancer patients with bone metastatic disease (FIG. 4I), but not within lymph node metastases (FIG. 4H).

In summary, in embodiments of the invention RAGE-expressing tumor cells (i) can arise focally in primary tumors and (ii) provide at least part of the apparently advantageous setting for bone marrow metastases. One can determine whether the observed prevalence of RAGE-positive tumor cells in human bone metastases represents selection (either an advantage in homing to bone marrow or a disadvantage in homing to other potential metastatic sites), induction (mediated by the bone marrow, but not other microenvironments), or a combination of mechanisms.

### Example 6

### Significance of Certain Embodiments of the Invention

A ligand/receptor-based molecular map of human blood vessels has been initiated, leading to mechanistic insights, and toward the basis of a targeted vascular pharmacology. A first patient screening (Arap et al., 2002) served as the foundation for an ongoing clinical trial of a new targeted drug lead, and the quantitative and qualitative methodologies (Kolonin et al., 2006a; Dias-Neto et al., 2009) have been markedly improved in an ethics framework that includes a set of cancer center-specific guidelines (Pentz et al., 2003) and a set of nationally appropriate recommendations (Pentz et al., 2005) to harmonize this line of patient-oriented research with the current practice of transplantation medicine. It is clear that large-scale accessibility of protein interactions in blood vessels of distinct organs is useful to expand the knowledge of many unidentified or poorly characterized molecular networks functioning at any given time in the human body. This leads to a considerably improved understanding of vascular proteomics and the generation of a new ligand-directed pharmacology with broad applications.

The inventors have also designed and developed a software filter to detect functional targeting peptides selected in three rounds of screening from patient biopsies obtained after intravenous administration of a phage library. Approximately 2.35×10⁶ motifs were generated, which comprised most of the displayed peptides in the tissues studied. Biostatistical analysis revealed a set of distinct tripeptides with preferential enrichment in specific tissues. Of those, four ligand-receptor systems were validated functionally, in shared or tissue-specific settings.

The inventors uncovered two tissue-specific vascular targeting systems. ANXA2 and prohibitin were found as a ligand-receptor pair in human WAT vasculature. Other studies with the entirely different yeast two-hybrid methodology have confirmed ANXA2 and prohibitin as interacting components in lipid rafts (Liu et al., 2005; Bacher et al., 2002). Given the demonstration of marked weight loss in obese rodents (Kolonin et al., 2004; Kim et al., 2010) and in obese monkeys (Barnhart et al. submitted) by the targeting of prohibitin in the vasculature with a peptide-guided pro-apoptotic peptidomimetic, in certain embodiments of the invention the selective mapping of this protein in human WAT vasculature and the confirmation of this molecular target has translational value as an investigational new drug lead in obese patients.

Furthermore, the inventors also demonstrate RAGE and proteinase-3 (PR-3) as molecular partners in human tumor-containing bone marrow resulting from primary tumorigenesis or metastasis. RAGE and PR-3 appeared unexpectedly as a molecular complex, apparently mediating the homing of human metastatic prostate cancer cells to the bone marrow. These proteins have, until now, been considered to be active in unrelated pathways and therefore, functionally distinct. PR-3 is abundantly secreted by activated bone marrow-derived polymorphonuclear leukocytes (Campanelli et al., 1990; Sturrock et al., 1992) and is deposited on the surface of endothelial cells as a result of tissue inflammation (Uehara et al., 2004; Zhou et al., 2000) activating a diverse range of targets in the myeloid microenvironment (Skold et al., 1999), and perhaps fostering leukocyte migration through basement membranes (Henson and Johnson, 1987). Given the additional data presented here, PR-3 also is functionally relevant to bone marrow-specific tumorigenesis and metastasis, in aspects of the invention.

In summary, it is clear that a large-scale analysis of protein interactions in particular tissues of healthy and diseased organs can uncover many presently unidentified or poorly characterized molecular networks.

### Example 7

### Materials and Methods

Reagents. The following reagents were used: mouse monoclonal anti-PR-3 antibody (Lab Vision and Accurate Chemicals); goat anti-RAGE IgG (R&D Systems), goat anti-ANXA2 IgG (Santa Cruz Biotechnology), and goat anti-GST IgG (Amersham Biosciences); rabbit anti-prohibitin IgG (Research Diagnostics), rabbit anti-caveolin-1 IgG (Santa Cruz Biotechnology). Secondary antibodies used were as follows: rat and goat anti-rabbit (Bio-Rad) or rat anti-goat (Promega) alkaline phosphatase-conjugated IgG; goat anti-rabbit horseradish peroxidase (HRP)-conjugated IgG (Sigma), and rabbit anti-human HRP-conjugated IgG (Sigma). The following recombinant proteins were used: His₆-ANXA2 and A5 (AmProx), stem cell growth factor alpha (SCGF-alpha) (Cell Sciences), ANXA1, and ANXAS (Novus Biologicals), PR-3 (Sigma), and RAGE-Fc and BMPRIA-Fc (R&D Systems), ApoE4, and ApoC (Sigma), and VEGFR (R&D Systems). GST-prohibitin was a gift from Dr. Srikumar Chellappan (H. Lee Moffitt Cancer Center & Research Institute). Human placentas were purchased from ILSbio. Human paraffin-embedded tissue samples (prostate, brain, fat, skin, and muscle) were obtained either from ILSbio or from an institution-banked panel of formalin-fixed samples (David H. Koch Center, The University of Texas M. D. Anderson Cancer Center).

Patient Selection and Clinical Course. This study adheres strictly to current medical ethics recommendations and guidelines regarding human research, and it has been reviewed and approved by the Clinical Ethics Service, the Institutional Biohazard Committee, Clinical Research Committee, and the Institutional Review Board of the University of Texas M. D. Anderson Cancer Center.

Patient #1 entered in the study was a 48-year-old Caucasian man with Waldenström macroglobulinemia who met the formal criteria for brain-based determination of death (Pentz et al., 2003; Pentz et al., 2005). Clinical attributes and detailed course of this human subject were reported elsewhere (Arap et al., 2002).

Patient #2 was a 66-year-old Caucasian man that presented with castration-resistant prostate cancer and predominant bone metastases. Six years prior to study entry, the primary tumor was diagnosed as a Gleason Score 10 (5+5) prostate cancer. Over his clinical course, the patient was treated with combined androgen ablation with the luteinizing hormone-releasing hormone (LHRH) antagonist leuprolide plus the antiandrogen bicalutamide. Several regimens of systemic chemo-hormonal therapy (ketoconazole plus doxorubicin alternating with viblastine plus estramustine; cyclophosphamide, vincristine, plus dexamethasone; docetaxel plus carboplatinum; and paclitaxel plus diethylstilbestrol or thalidomide; vinorelbine; mitoxantrone; PC-SPES), radiopharmaceutical therapy (Strontium-89), and targeted therapy with a proteasome inhibitor (bortezomib) were given sequentially over time. Patient #2 also underwent courses of external beam radiation therapy for bone pain palliation in the neck (3,000 cGray, C1-T2) and pelvic (3,000 cGray, L2-S1) metastatic sites. Ultimately, Patient#2 presented to the emergency room with respiratory and cardiovascular failure secondary to worsening pleural effusion and hemothorax. Despite thoracocentesis, endotracheal intubation, mechanical ventilation, and full medical support in an intensive care unit setting, he evolved into multiple organ failure. Based on his irreversible clinical condition, a terminal wean from life-support systems was planned in accordance to previously stated patient wishes. After discussion with the family and a surrogate informed written consent was obtained from legal next-of-kin, the patient was enrolled in the study.

Patient #3 was a 73-year-old Caucasian man that presented with locally advanced prostate cancer. Two years prior to study entry, he was diagnosed with Gleason Score 9 (4+5) prostate cancer and treated with integrated external bean radiation therapy plus brachytherapy implants and long-term androgen ablation with the LHRH antagonist leuprolide. He subsequently developed castration-resistant prostate cancer with predominant bone metastases. He was treated with systemic chemotherapy (docetaxel plus prednisone) and a course of external beam radiation therapy for palliation of bone metastasis pain in the lumbar spine (3,000 cGray, L1-L5). Previously to the diagnosis of prostate cancer, the patient had been successfully treated for a non-Hodgkin lymphoma (diffuse large cell type involving head and neck) with systemic chemo-immunotherapy (cyclophosphamide, doxorubicin, vincristine, prednisone, and rituximab) plus mantle radiation therapy. Nine years later, at the time of study entry, he had no clinical or laboratory evidence of lymphoma and was presumably cured from that tumor. Patient #3 had multiple co-morbidities including arterial hypertension, coronary artery disease (status post several coronary artery bypass graft surgeries and vascular stent insertion procedures), plus radiation-induced lung fibrosis. During an inpatient admission for worsening chest and abdominal pain, he developed severe acute respiratory distress syndrome and was transferred to the intensive care unit but became critically ill and unresponsive under prolonged endotracheal intubation and mechanical ventilation. Based on this clinically irreversible condition, a terminal wean from life-support systems was requested. Thus, after informed written consent was obtained from the patient and the legal next-of-kin, the patient was enrolled in the study.

Administration of Phage Display Library and Sample Collection. Endotoxin levels of administered random peptide libraries were assessed with Endosafe (Charles River). Short-term intravenous infusion of phage display sub-library recovered from the first, second and third rounds of selection (Arap et al., 2002) (2×10¹² TU from each organ; total 10¹³ TU pooled) were followed by multiple representative tissue biopsies. Prostate, liver, and metastatic tumor samples were obtained by needle biopsy under ultrasonographic guidance; skin, adipose tissue, and skeletal muscle samples were obtained surgically. Bone marrow needle aspirates and core biopsy samples were also obtained. After systemic delivery of a naïve phage-displayed random peptide library to the first human subject (Arap et al., 2002), ligand phage populations were recovered, pooled, and serially screened in the two subsequent patients.

Post-biopsy Processing of Human Tissue Samples. Universal precautions were used by the laboratory personnel handling human samples. The amount of phage present in each tissue was determined by either TU-counting (Pasqualini and Ruoslahti, 1996; Arap et al., 2002) and/or quantitative real-time PCR (Dias-Neto et al., 2009). The PCR reaction admixture consisted of 60 ng of total DNA, Power SYBR Green PCR Master Mix (Applied Biosystems), and 3.75 picomoles of oligonucleotide primers directed to the amplification of a fragment of the fUSES pIII gene. For each experiment, standard curves were generated with serial dilutions of phage plasmid, from 2.4×10² to 2.4×10⁶ copies. Each point on the curve, as well as each tissue sample of DNA, was determined in triplicates. A standard calibration curve was calculated by the Applied Biosystems 7500 Fast System SDS software (version 1.3.1.21, Applied Biosystems) through regression of the crossing points of the PCR curves from plasmid dilutions. The number of viral particles in each DNA sample was determined by comparison of the amplification threshold for each sample to the standard curve. The amplification efficiency (AE) of each PCR cycle was calculated from the slope (s) of the standard curve through the equation AE=10¹/(^{-s}). All amplifications and calculations were performed with an ABI7500 Fast system (Applied Biosystems). For large-scale sequencing, total DNA was extracted and used for PCR amplification of phage inserts. The amplicons produced from tissues and the CX₇C library were subsequently purified and sequenced with a pyrosequencing approach (FLX platform, Roche/454).

Statistical Analysis. One-sided Fisher's exact test was used to identify tripeptide motifs significantly enriched after three rounds of selection, for each targeted tissue, and for comparison to the parental unselected random peptide library. A Monte Carlo algorithm (Kolonin et al., 2006a) was applied to minimize the number of assumptions, and to account for the large number of comparisons made for each round. Simulations were generated and a “computational staining plot” was produced for each targeted tissue at each round of selection, after comparison to the random peptide library and to unrelated tissues. Analysis of peptide sequences was executed with a character pattern recognition program based on SAS (version 8.1.2; SAS Institute) and Perl (version 5.8.1). To identify peptide similarities to human proteins, the inventors codified Peptide Match software in Perl 5.8.1 based on RELIC (Arap et al., 2002). Peptide-protein similarity scores for each residue were calculated based on a modified BLOSUM62 substitution matrix.

Peptide Synthesis and Antibody Production. The peptides CWELGGGPC (SEQ ID NO:2), CPGGGLVHC (SEQ ID NO:6), CKGGRAKDC (SEQ ID NO:1), and a negative control peptide (sequence CARAC (SEQ ID NO:7), unless otherwise specified) were chemically synthesized, cyclized, tagged on the N-terminus with biotin or KLH, and purified by high-performance liquid chromatography (HPLC) by commercial vendors (AnaSpec, Genemed Synthesis, PolyPeptide Laboratories, or Sigma). Antisera against cyclized KLH-conjugated peptides were produced in rabbits.

Protein Extraction and Peptide Affinity Chromatography. Human tissue samples were homogenized in ice-cold tris-buffered saline (TBS) supplemented with 100 mM phenylmethylsulfonyl fluoride (PMSF). Following extensive washes, tissue pellets were resuspended in extraction buffer (TBS containing 100 mM octylglucoside, 100 mM PMSF, 10 mM CaCl₂, and 10 mM MgCl₂), and protein extraction was carried out overnight (ON) at 4° C. Three cycles of extraction were performed. Membrane proteins of human white mononuclear bone marrow cells were purified on a Ficoll gradient. Isolation of membrane proteins from WAT and their separation into caveolar and non-caveolar lipid raft fractions were based on established protocols (Smart et al., 1995). Extracted proteins were chromatographed on affinity columns (Pierce) previously conjugated with each synthetic peptide of interest. Columns were washed extensively and were eluted with a solution of the corresponding peptide followed by a low pH buffer (extraction buffer supplemented with 0.1 M glycine and 0.1 M NaCl, pH 2.5). Fractions of 0.5 ml were collected, and those containing protein (O.D. 280 nm) were used for further studies.

Mass Spectrometry. Protein identification was carried out through a Nano LC-MS/MS peptide sequencing technology (ProtTech). In brief, each protein gel band was destained, cleaned, and digested in-gel with sequencing grade modified trypsin. The resulted peptide mixture was analyzed by a LC-MS/MS system, in which a HPLC with a 75 μm inner diameter reverse-phase C18 column was on-line coupled to an ion-trap mass spectrometer. The mass spectrometric data acquired were used to search a non-redundant protein database. The output from the database search was manually validated before reporting. The following peptides were identified: PR-3, LFPDFFTRVAYVDWIR (SEQ ID NO:8), LVNVVLGAHNVRTQEPTQQHFSVAQVFLNNYDAENK (SEQ ID NO:9), and IVGGHEAQPHSRPYMASLQMR (SEQ ID NO:10).

Protein Microarray Screening. High-density arrays of the protein expression set of the hEx1 library were commercially obtained (imaGenes). For rabbit anti-peptide serum profiling, the filters were blocked in 2% (w/v) non-fat, dry milk powder in TBST [TBS containing 0.1% (v/v) Tween-20] for 2 h, washed twice in TBST, and subsequently incubated with anti-peptide serum diluted 1:1,000 for 16 h. Following three 30 min TBST washes and subsequent incubation with the secondary antibody (anti-rabbit-IgG-alkaline phosphatase, Sigma) at 1:5,000 dilution in 2% (w/v) milk/TBST, the filters were washed three times in TBST-T for 20 min each, followed by a 10 min wash in TBS and a further wash for 10 min in alkaline phosphatase buffer (1 mM MgCl₂, 0.1 M Tris pH 9.5), and subsequent incubation in 25 mM Attophos (Roche) in alkaline phosphatase buffer for 5 min. The filters were illuminated with long-wave ultra-violet light, and the images were taken with a high-resolution CCD detection system (Fuji). Image analysis was performed with VisualGrid (GPC Biotech). Positive clone cDNA inserts were amplified and sequenced for identity confirmation of expressed proteins.

Phage Binding Assays. Binding of targeted phage to immobilized candidate receptors was evaluated as described (Kolonin et al., 2006b). Micro-wells of 96-well plates were blocked with phosphate-buffered saline (PBS) containing 3% BSA, washed, and incubated with 10⁹ TU of targeted phage. Inhibition of phage binding was performed in the presence of increasing concentrations of synthetic peptides, as indicated. For phage display screening on immobilized prohibitin, 10⁹ TU of phage clones recovered from the second round of in vivo selection were incubated ON with 1 μg of immobilized recombinant GST-prohibitin. Bound phage were recovered by infection of host bacteria (E. coli K91 Kan).

Protein Binding Assays. Titration of anti-peptide antibodies was performed on Maxisorb Immunoplates (Nunc) coated with 1 μg/mL of peptides or proteins. Incubation with primary antibodies was followed by signal detection with goat anti-rabbit HRP-conjugated IgG (Sigma-Aldrich; St. Louis, Mo.) and 3, 3′,5,5′-tetramethylbenzidine (TMB) (Calbiochem). To evaluate protein-protein interactions, the inventors performed ELISA on 96-well plates coated with 1 μg/mL of recombinant candidate receptors, as indicated. Blocking of exposed non-specific binding sites was performed with PBS containing either 2% gelatin or 1% BSA, as indicated. Ligand candidates were added to the wells at different concentrations, as indicated. Specific binding was detected by incubation with appropriate primary and secondary antibodies. For capture experiments, immobilized His₆-ANXA2 and ANXAS were incubated with recombinant GST-prohibitin. Protein interaction, assessed by immunoblotting with anti-GST antibody, was detected with anti-rabbit or anti-goat secondary alkaline phosphatase-conjugated polyclonal antibodies.

Immunostaining. Immunohistochemical staining of normal human TMAs (CelleStan) was performed as follows. After complete removal of paraffin and antigen retrieval in high pH, slides were incubated with primary antibodies followed by appropriate HRP-conjugated secondary antibodies (EnVision DakoCytomation or Vector). High-resolution pictures were obtained with ImageScope (Aperio) Immunohistochemical staining of bone marrow and prostate cancer specimens was performed on 4 μm sections and carried out either in an Autostainer or manually. When required, antigen retrieval was performed with target retrieval solution (Dako). Tissue sections were incubated with primary antibody for 1 h, and the reactions were developed with either the labeled streptavidin-biotin (LSAB) system or the EnVision kit (Dako). Sections were counterstained with hematoxylin (Biocare Medical).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims. The entire contents of any reference that is referred to herein are hereby incorporated by reference.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 5,021,236
U.S. Pat. No. 5,446,137
U.S. Pat. No. 5,614,617
U.S. Pat. No. 5,652,099
U.S. Pat. No. 5,670,663
U.S. Pat. No. 5,672,697
U.S. Pat. No. 5,681,947
U.S. Pat. No. 5,714,606
U.S. Pat. No. 5,763,167
U.S. Pat. No. 5,859,221
U.S. Pat. No. 5,872,232
U.S. Pat. No. 5,886,165
Arap W, et al. (2002) Steps toward mapping the human vasculature by phage display. Nat Med 8:121-127.
Bacher S, Achatz G, Schmitz M L, Lamers M C (2002) Prohibitin and prohibitone are contained in high-molecular weight complexes and interact with alpha-actinin and annexin A2. Biochimie 84:1207-1220.
Barrios-Rodiles M, et al. (2005) High-throughput mapping of a dynamic signaling network in mammalian cells. Science 307:1621-1625.
Benincasa, M. et al., Rapid and Reliable Detection of Antimicrobial Peptide Penetration into Gram-Negative Bacteria Based on Fluorescence Quenching, Antimicrobial Agents and Chemotherapy, August 2009, p. 3501-3504
Breitkreutz A, et al. (2010) A global protein kinase and phosphatase interaction network in yeast. Science 328:1043-1046.
Campanelli D, et al. (1990) Cloning of cDNA for proteinase 3: a serine protease, antibiotic, and autoantigen from human neutrophils. J Exp Med 172:1709-1715.
Cardo-Vila, M. et al., Alpha v beta 5 integrin-dependent programmed cell death triggered by a peptide mimic of annexin V, Mol Cell. 2003 May; 11(5):1151-62.
Denli, Trends Biochem. Sci 28: 196, 2003.
Dias-Neto E, et al. (2009) Next-generation phage display: integrating and comparing available molecular tools to enable cost-effective high-throughput analysis. PLoS One 4:e8338 1-11.
Folkman J (2007) Angiogenesis: an organizing principle for drug discovery? Nat Rev Drug Discov 6:273-286.
Gorfu G, Rivera-Nieves J, Ley K (2009) Role of 7 integrins in intestinal lymphocyte homing and retention. Curr Mol Med 9:836-850.
Hatters D M, Peters-Libeu C A, Weisgraber K H (2006) Apolipoprotein E structure: insights into function. Trends Biochem Sci 31:445-454.
Henriques S T, Melo M N, Castanho M A. How to address CPP and AMP translocation? Methods to detect and quantify peptide internalization in vitro and in vivo (Review). Mol Membr Biol. 2007 May-June; 24(3):173-84.
Henson P M, Johnston R B Jr (1987) Tissue injury in inflammation. Oxidants, proteinases, and cationic proteins. J Clin Invest 79:669-674.
Huttunen H J, Fages C, Kuja-Panula J, Ridley A J, Rauvala H (2002)
Receptor for advanced glycation end products-binding COOH-terminal motif of amphoterin inhibits invasive migration and metastasis. Cancer Res 62:4805-4811.
Ishiguro H, et al. (2005) Receptor for advanced glycation end products (RAGE) and its ligand, amphoterin are overexpressed and associated with prostate cancer development. Prostate 64:92-100.
Jeon Y J, et al. (2010) Annexin A4 interacts with NF-kappaB p50 subunit and modulates NF-kappaB transcriptional activity in a Ca+-dependent manner. Cell Mol Life Sci 67:2271-2281.
Jiao, C.-Y. et al., Translocation and endocytosis for cell-penetrating peptide internalization, J. Biol. Chem., vol. 284, NO. 49, pp. 33957-33965, Dec. 4, 2009.
Kim D H, Woods S C, Seeley R J (2010) Peptide designed to elicit apoptosis in adipose tissue endothelium reduces food intake and body weight. Diabetes 59:907-915.
Koivunen E, et al. (2001) Inhibition of β₂ integrin-mediated leukocyte cell adhesion by leucine-leucine-glycine motif-containing peptides. J Cell Biol 153:905-916.
Kolonin M G, et al. (2006b) Ligand-directed surface profiling of human cancer cells with combinatorial peptide libraries. Cancer Res 66:34-40.
Kolonin M G, et al. (2006a) Synchronous selection of homing peptides for multiple tissues by in vivo phage display. FASEB J 20:979-981.
Kolonin M G, Saha P K, Chan L, Pasqualini R, Arap W (2004) Reversal of obesity by targeted ablation of adipose tissue. Nat Med 10:625-632.
Ling Q, et al. (2004) Annexin II regulates fibrin homeostasis and neoangiogenesis in vivo. J Clin Invest 113:38-48.
Liu J, Deyoung S M, Zhang M, Dold L H, Saltiel A R (2005) The stomatin/prohibitin/flotillin/HflK/C domain of flotillin-1 contains distinct sequences that direct plasma membrane localization and protein interactions in 3T3-L1 adipocytes. J Biol Chem 280:16125-16134.
Lobb R R, Hemler M E (1994) The pathophysiologic role of 4 integrins in vivo. J Clin Invest 94:1722-1728.
Lutgens S P M, Cleutjens K B, Daemen M J, Heeneman S (2007) Cathepsin cysteine proteases in cardiovascular disease. FASEB J 21:3029-3041.
Molldrem J J, et al. (2000) Evidence that specific T lymphocytes may participate in the elimination of chronic myelogenous leukemia. Nat Med 6:1018-1023.
Nakase, I., et al., Cellular uptake of arginine-rich peptides: roles for macropinocytosis and actin rearrangement, Molecular Therapy (2004) 10, 1011-1022.
Olsen, et al., Dev. Biol. 216:671, 1999.
Pasqualini R, et al. (2000) Aminopeptidase N is a receptor for tumor-homing peptides and a target for inhibiting angiogenesis. Cancer Res 60:722-727.
Pasqualini R, Ruoslahti E (1996) Organ targeting in vivo using phage display peptide libraries. Nature 380:364-366.
Pentz R D, et al. (2005) Ethics guidelines for research with the recently dead. Nat Med 11:1145-1149.
Pentz R D, Flamm A L, Pasqualini R, Logothetis C J, Arap W (2003) Revisiting technical guidelines for research with terminal wean and brain-dead patients. Hastings Cent Rep 33:20-26.
Risau W, Flamme I (1995) Vasculogenesis. Annu Rev Cell Dev Biol 11:73-91.
Rose D M, Alon R, Ginsberg M H (2007) Integrin modulation and signaling in leukocyte adhesion and migration. Immunol Rev 218:126-134.
Rual J F, et al. (2005) Towards a proteome-scale map of the human protein-protein interaction network. Nature 437:1173-1178.
Seggerson, et al., Dev. Biol. 243: 215, 2002.
Sergeeva A, Kolonin M G, Molldrem J J, Pasqualini R, Arap W (2006) Display technologies: application for the discovery of drug and gene delivery agents. Adv Drug Deliv Rev 58:1622-1654.
Skold S, Rosberg B, Gullberg U, Olofsson T A (1999) Secreted proform of neutrophil proteinase 3 regulates the proliferation of granulopoietic progenitor cells. Blood 93:849-856.
Smart E J, Ying Y S, Mineo C, Anderson R G (1995) A detergent-free method for purifying caveolae membrane from tissue culture cells. Proc Natl Acad Sci USA 92:10104-10108.
Smith G P (1985) Filamentous fusion phage: novel expression vectors that display cloned antigens on the virion surface. Science 228:1315-1317.
Smith G P, Scott J K (1993) Libraries of peptides and proteins displayed on filamentous phage. Methods Enzymol 217:228-257.
Staquicini F I, Pasqualini R, Arap W (2009) Ligand-directed profiling: applications to target drug discovery in cancer. Exp Opin Drug Disc 4:51-59.
Stelzl U, et al. (2005) A human protein-protein interaction network: a resource for annotating the proteome. Cell 122:957-968.
Sturrock A B, et al. (1992) Structure, chromosomal assignment, and expression of the gene for proteinase-3. The Wegener's granulomatosis autoantigen. J Biol Chem 267:21193-21199.
Tonelli R R, et al. (2010) Role of the gp85/trans-sialidases in trypanosoma cruzi tissue tropism: preferential binding of a conserved peptide motif to the vasculature in vivo. PLoS Negl Trop Dis 4:e864.
Uehara A, Sugawara Y, Sasano T, Takada H, Sugawara S (2004) Proinflammatory cytokines induce proteinase 3 as membrane-bound and secretory forms in human oral epithelial cells and antibodies to proteinase 3 activate the cells through protease-activated receptor-2. J Immunol 173:4179-4189.
Vendruscolo M, Paci E, Dobson C M, Karplus M (2001) Three key residues form a critical contact network in a protein folding transition state. Nature 409:641-645.
Zetter B R (1990) The cellular basis of site-specific tumor metastasis. N Engl J Med 322: 605-612.
Zhang J, McCrae K R (2005) Annexin A2 mediates endothelial cell activation by antiphospholipid/anti-beta2 glycoprotein I antibodies. Blood 105:1964-1969.
Zhou Z, Dionne A, Richard C, Menard H A (2000) On the origin of surface proteinase 3 of nonmyeloid cells: evidence favoring an exogenous source. Clin Immunol 97:171-181.
Zurita A J, et al. (2004) Combinatorial screenings in patients: the interleukin-11 receptor alpha as a candidate target in the progression of human prostate cancer. Cancer Res 64:435-439.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1.-114. (canceled)

115. A targeting agent characterized in that it interacts specifically with a targeted entity that is or comprises prohibitin, wherein the targeting agent is not a peptide consisting of an amino acid sequence of SEQ ID NO: 1.

116. The targeting agent of claim 115, wherein the targeting agent is or comprises a peptide.

117. The targeting agent of claim 115, wherein the targeting agent is or comprises a peptide comprising an amino acid sequence selected from the group consisting of SEQ ID NOS: 14-24, 44-104, and a combination thereof.

118. The targeting agent of claim 116, wherein the peptide has amino acid sequence of 80% or more overall identity with a portion of annexin A2 (ANXA2) that is at least 3 amino acids in length.

119. The targeting agent of claim 116, wherein the peptide includes an amino acid moiety selected from the group consisting of SEQ ID NOS: 14-24, 44-104 and a combination thereof and wherein the targeting agent is not ANXA2.

120. The targeting agent of claim 115, wherein the targeting agent is linked with a molecule or an active agent.

121. The targeting agent of claim 120, wherein the active agent is selected from the group consisting of a diagnostic agent and a therapeutic agent.

122. The targeting agent of claim 115, wherein the targeting agent is or comprises a peptide of 3-30 residues in length.

123. The targeting agent of claim 115, characterized in that, when it is contacted with a system comprising a targeted entity and a targeting peptide that interacts specifically with the targeted entity, the targeting agent competes with the targeting peptide for interaction with the targeted entity.

124. A method of delivering a targeting agent to a targeted entity site, the method comprising step of: delivering to the targeted entity site a targeting agent of claim 115.

125. The method of claim 124, wherein the targeting agent is or comprises a peptide.

126. The method of claim 125, wherein the targeting agent is or comprises a peptide comprising an amino acid sequence selected from the group consisting of SEQ ID NOS: 14-24, 44-104, and a combination thereof.

127. The method of claim 125, wherein the targeting agent comprises a peptide whose amino acid sequence has at least 80% overall identity with a portion of ANXA2 that is at least 3 amino acids in length, and further includes an amino acid moiety as set forth in SEQ ID NOS: 14-24, 44-104, or a combination thereof, wherein the targeting agent is not ANXA2.

128. The method of claim 124, wherein the targeting agent is linked with an active agent.

129. The method of claim 128, wherein the active agent is selected from the group consisting of a diagnostic agent and a therapeutic agent.
